SEQUENCE LISTING (186 sequences)

SEQ ID NO: 1 - RNA, 67 nt, Artificial Sequence
  Description of Artificial Sequence Synthetic oligonucleotide
agcauagcaaguuaaaauaaggcuaguccguuaucaacuugaaaaaguggcaccgagucggugcuuu

SEQ ID NO: 2 - RNA, 36 nt, Artificial Sequence
  Description of Artificial Sequence Synthetic oligonucleotide
cucgcagcucaccaguuuuagagcuaugcuguuuug

SEQ ID NO: 3 - RNA, 36 nt, Artificial Sequence
  Description of Artificial Sequence Synthetic oligonucleotide
gcggcuggagguggguuuuagagcuaugcuguuuug

SEQ ID NO: 4 - RNA, 36 nt, Artificial Sequence
  Description of Artificial Sequence Synthetic oligonucleotide
cucuaugguggcguguuuuagagcuaugcuguuuug

SEQ ID NO: 5 - RNA, 36 nt, Artificial Sequence
  Description of Artificial Sequence Synthetic oligonucleotide
cuccaccccgccucguuuuagagcuaugcuguuuug

SEQ ID NO: 6 - RNA, 36 nt, Artificial Sequence
  Description of Artificial Sequence Synthetic oligonucleotide
gcuauucucgcagcucaccaguuuuagagcuaugcu

SEQ ID NO: 7 - RNA, 36 nt, Artificial Sequence
  Description of Artificial Sequence Synthetic oligonucleotide
ggcacugcggcuggagguggguuuuagagcuaugcu

SEQ ID NO: 8 - RNA, 36 nt, Artificial Sequence
  Description of Artificial Sequence Synthetic oligonucleotide
gacuuccucuaugguggcguguuuuagagcuaugcu

SEQ ID NO: 9 - RNA, 36 nt, Artificial Sequence
  Description of Artificial Sequence Synthetic oligonucleotide
gacccccuccaccccgccucguuuuagagcuaugcu

SEQ ID NO: 10 - DNA, 30 bp, Artificial Sequence
  Description of Artificial Sequence Synthetic oligonucleotide
cccaagaagaagaggaaagtctaataattg

SEQ ID NO: 11 - DNA, 22 bp, Artificial Sequence
  Description of Artificial Sequence Synthetic oligonucleotide
gtgatggtgatgatgatgactg

SEQ ID NO: 12 - DNA, 49 bp, Artificial Sequence
  Description of Artificial Sequence Synthetic oligonucleotide
gggcagcagtcatcatcatcaccatcacccaaagaagaagcggaaagtc

SEQ ID NO: 13 - DNA, 43 bp, Artificial Sequence
  Description of Artificial Sequence Synthetic oligonucleotide
attagactttcctcttcttcttgggctcgaattcgctgccgtc

SEQ ID NO: 14 - DNA, 43 bp, Artificial Sequence
  Description of Artificial Sequence Synthetic oligonucleotide
catggacgagctgtacaagggatccggcggcctaaggactcgg

SEQ ID NO: 15 - DNA, 39 bp, Artificial Sequence
  Description of Artificial Sequence Synthetic oligonucleotide
ctgaagttagtagctccgctgaagccggccttgcacatg

SEQ ID NO: 16 - DNA, 18 bp, Artificial Sequence
  Description of Artificial Sequence Synthetic oligonucleotide
agcggagctactaacttc

SEQ ID NO: 17 - DNA, 47 bp, Artificial Sequence
  Description of Artificial Sequence Synthetic oligonucleotide
ggtcggcgcgcccacccttggatcctcacttgtacagctcgtccatg

SEQ ID NO: 18 - DNA, 18 bp, Artificial Sequence
  Description of Artificial Sequence Synthetic primer
tggcggcctaaggactcg

SEQ ID NO: 19 - DNA, 22 bp, Artificial Sequence
  Description of Artificial Sequence Synthetic primer
cttcagggtgaggatgcctctc

SEQ ID NO: 20 - DNA, 21 bp, Artificial Sequence
  Description of Artificial Sequence Synthetic primer
ccagtggttcaatggtcatcc

SEQ ID NO: 21 - DNA, 19 bp, Artificial Sequence
  Description of Artificial Sequence Synthetic primer
ggccagtgaaatcaccctg

SEQ ID NO: 22 - DNA, 19 bp, Artificial Sequence
  Description of Artificial Sequence Synthetic primer
gtttccgaggcagcagttg

SEQ ID NO: 23 - DNA, 21 bp, Artificial Sequence
  Description of Artificial Sequence Synthetic primer
gcatgataaacgtcatcgccc

SEQ ID NO: 24 - DNA, 22 bp, Artificial Sequence
  Description of Artificial Sequence Synthetic primer
agagaagtcgaggaagagagag

SEQ ID NO: 25 - DNA, 22 bp, Artificial Sequence
  Description of Artificial Sequence Synthetic primer
cagcagaaagttcatggtttcg

SEQ ID NO: 26 - DNA, 51 bp, Artificial Sequence
  Description of Artificial Sequence Synthetic primer
tcgtcggcagcgtcagatgtgtataagagacagtggcggcctaaggactcg

SEQ ID NO: 27 - DNA, 53 bp, Artificial Sequence
  Description of Artificial Sequence Synthetic primer
gtctcgtgggctcggagatgtgtataagagacaggaagccggccttgcacatg

SEQ ID NO: 28 - DNA, 53 bp, Artificial Sequence
  Description of Artificial Sequence Synthetic primer
tcgtcggcagcgtcagatgtgtataagagacagggtccaaagcaggatgacag

SEQ ID NO: 29 - DNA, 53 bp, Artificial Sequence
  Description of Artificial Sequence Synthetic primer
gtctcgtgggctcggagatgtgtataagagacaggagacacacacacaggcct

SEQ ID NO: 30 - DNA, 52 bp, Artificial Sequence
  Description of Artificial Sequence Synthetic primer
tcgtcggcagcgtcagatgtgtataagagacagcactgctctccagagtggt

SEQ ID NO: 31 - DNA, 56 bp, Artificial Sequence
  Description of Artificial Sequence Synthetic primer
gtctcgtgggctcggagatgtgtataagagacaggattctcctacttcctcctcgg

SEQ ID NO: 32 - DNA, 54 bp, Artificial Sequence
  Description of Artificial Sequence Synthetic primer
tcgtcggcagcgtcagatgtgtataagagacaggagaaaagccaacgggttctc

SEQ ID NO: 33 - DNA, 56 bp, Artificial Sequence
  Description of Artificial Sequence Synthetic primer
gtctcgtgggctcggagatgtgtataagagacagcattgtcccagctaagctctca

SEQ ID NO: 34 - DNA, 56 bp, Artificial Sequence
  Description of Artificial Sequence Synthetic primer
tcgtcggcagcgtcagatgtgtataagagacagccctgagaaggtagtaggaatcc

SEQ ID NO: 35 - DNA, 55 bp, Artificial Sequence
  Description of Artificial Sequence Synthetic primer
gtctcgtgggctcggagatgtgtataagagacaggtggttaagagcagactccct

SEQ ID NO: 36 - DNA, 50 bp, Artificial Sequence
  Description of Artificial Sequence Synthetic primer
tcgtcggcagcgtcagatgtgtataagagacagctcctccgaagcgagaa

SEQ ID NO: 37 - DNA, 53 bp, Artificial Sequence
  Description of Artificial Sequence Synthetic primer
gtctcgtgggctcggagatgtgtataagagacaggacagacagacagacaccg

SEQ ID NO: 38 - DNA, 56 bp, Artificial Sequence
  Description of Artificial Sequence Synthetic primer
tcgtcggcagcgtcagatgtgtataagagacaggctctgaccttgtttgttattcc

SEQ ID NO: 39 - DNA, 53 bp, Artificial Sequence
  Description of Artificial Sequence Synthetic primer
gtctcgtgggctcggagatgtgtataagagacaggtgactccaaggcttttcg

SEQ ID NO: 40 - DNA, 50 bp, Artificial Sequence
  Description of Artificial Sequence Synthetic primer
tcgtcggcagcgtcagatgtgtataagagacagctcttgcctgtcacgca

SEQ ID NO: 41 - DNA, 54 bp, Artificial Sequence
  Description of Artificial Sequence Synthetic primer
gtctcgtgggctcggagatgtgtataagagacagcctggagttaagggtgtctc

SEQ ID NO: 42 - DNA, 53 bp, Artificial Sequence
  Description of Artificial Sequence Synthetic primer
tcgtcggcagcgtcagatgtgtataagagacagcatccttgtatcagctgcct

SEQ ID NO: 43 - DNA, 57 bp, Artificial Sequence
  Description of Artificial Sequence Synthetic primer
gtctcgtgggctcggagatgtgtataagagacagccattctcagcctaaaaggtaga

SEQ ID NO: 44 - DNA, 51 bp, Artificial Sequence
  Description of Artificial Sequence Synthetic primer
aatgatacggcgaccaccgagatctacacctctctattcgtcggcagcgtc

SEQ ID NO: 45 - DNA, 51 bp, Artificial Sequence
  Description of Artificial Sequence Synthetic primer
aatgatacggcgaccaccgagatctacactatcctcttcgtcggcagcgtc

SEQ ID NO: 46 - DNA, 51 bp, Artificial Sequence
  Description of Artificial Sequence Synthetic primer
aatgatacggcgaccaccgagatctacacgtaaggagtcgtcggcagcgtc

SEQ ID NO: 47 - DNA, 51 bp, Artificial Sequence
  Description of Artificial Sequence Synthetic primer
aatgatacggcgaccaccgagatctacacactgcatatcgtcggcagcgtc

SEQ ID NO: 48 - DNA, 51 bp, Artificial Sequence
  Description of Artificial Sequence Synthetic primer
aatgatacggcgaccaccgagatctacacaaggagtatcgtcggcagcgtc

SEQ ID NO: 49 - DNA, 51 bp, Artificial Sequence
  Description of Artificial Sequence Synthetic primer
aatgatacggcgaccaccgagatctacacctaagccttcgtcggcagcgtc

SEQ ID NO: 50 - DNA, 51 bp, Artificial Sequence
  Description of Artificial Sequence Synthetic primer
aatgatacggcgaccaccgagatctacaccgtctaattcgtcggcagcgtc

SEQ ID NO: 51 - DNA, 51 bp, Artificial Sequence
  Description of Artificial Sequence Synthetic primer
aatgatacggcgaccaccgagatctacactctctccgtcgtcggcagcgtc

SEQ ID NO: 52 - DNA, 47 bp, Artificial Sequence
  Description of Artificial Sequence Synthetic primer
caagcagaagacggcatacgagattcgccttagtctcgtgggctcgg

SEQ ID NO: 53 - DNA, 47 bp, Artificial Sequence
  Description of Artificial Sequence Synthetic primer
caagcagaagacggcatacgagatctagtacggtctcgtgggctcgg

SEQ ID NO: 54 - DNA, 47 bp, Artificial Sequence
  Description of Artificial Sequence Synthetic primer
caagcagaagacggcatacgagatttctgcctgtctcgtgggctcgg

SEQ ID NO: 55 - DNA, 47 bp, Artificial Sequence
  Description of Artificial Sequence Synthetic primer
caagcagaagacggcatacgagatgctcaggagtctcgtgggctcgg

SEQ ID NO: 56 - DNA, 47 bp, Artificial Sequence
  Description of Artificial Sequence Synthetic primer
caagcagaagacggcatacgagataggagtccgtctcgtgggctcgg

SEQ ID NO: 57 - DNA, 47 bp, Artificial Sequence
  Description of Artificial Sequence Synthetic primer
caagcagaagacggcatacgagatcatgcctagtctcgtgggctcgg

SEQ ID NO: 58 - DNA, 47 bp, Artificial Sequence
  Description of Artificial Sequence Synthetic primer
caagcagaagacggcatacgagatgtagagaggtctcgtgggctcgg

SEQ ID NO: 59 - DNA, 47 bp, Artificial Sequence
  Description of Artificial Sequence Synthetic primer
caagcagaagacggcatacgagatcagcctcggtctcgtgggctcgg

SEQ ID NO: 60 - DNA, 23 bp, Homo sapiens
  modified_base (21)..(21): a, c, t, g, unknown or other
ggcactgcggctggaggtggngg

SEQ ID NO: 61 - DNA, 23 bp, Homo sapiens
  modified_base (21)..(21): a, c, t, g, unknown or other
ggcacgacggctggaggtggngg

SEQ ID NO: 62 - DNA, 23 bp, Homo sapiens
  modified_base (21)..(21): a, c, t, g, unknown or other
ggcgctgcggcgggaggtggngg

SEQ ID NO: 63 - DNA, 23 bp, Homo sapiens
  modified_base (21)..(21): a, c, t, g, unknown or other
ggcactgcggctggaggtggngg

SEQ ID NO: 64 - DNA, 23 bp, Homo sapiens
  modified_base (21)..(21): a, c, t, g, unknown or other
tgcactgcggccggaggaggngg

SEQ ID NO: 65 - DNA, 23 bp, Homo sapiens
  modified_base (21)..(21): a, c, t, g, unknown or other
agcagtgcggctagaggtggngg

SEQ ID NO: 66 - DNA, 23 bp, Homo sapiens
  modified_base (21)..(21): a, c, t, g, unknown or other
ggcaatgcggctggaggcggngg

SEQ ID NO: 67 - DNA, 23 bp, Homo sapiens
  modified_base (21)..(21): a, c, t, g, unknown or other
ggcactggggttggaggtggngg

SEQ ID NO: 68 - DNA, 23 bp, Homo sapiens
  modified_base (21)..(21): a, c, t, g, unknown or other
ggcactgctgctgggggtggngg

SEQ ID NO: 69 - DNA, 23 bp, Homo sapiens
  modified_base (21)..(21): a, c, t, g, unknown or other
ggctctgcggctggagggggngg

SEQ ID NO: 70 - DNA, 23 bp, Homo sapiens
  modified_base (21)..(21): a, c, t, g, unknown or other
gacaccacggctggagatggngg

SEQ ID NO: 71 - DNA, 23 bp, Homo sapiens
  modified_base (21)..(21): a, c, t, g, unknown or other
ggcatcacggctggaggtggngg

SEQ ID NO: 72 - DNA, 23 bp, Homo sapiens
  modified_base (21)..(21): a, c, t, g, unknown or other
aggactgcggctgggggtggngg

SEQ ID NO: 73 - DNA, 23 bp, Homo sapiens
  modified_base (21)..(21): a, c, t, g, unknown or other
ggcactgagggtggaggtggngg

SEQ ID NO: 74 - DNA, 23 bp, Homo sapiens
  modified_base (21)..(21): a, c, t, g, unknown or other
ggcactgtggctgcaggtggngg

SEQ ID NO: 75 - DNA, 23 bp, Homo sapiens
  modified_base (21)..(21): a, c, t, g, unknown or other
cacactgcagctggaggtggngg

SEQ ID NO: 76 - DNA, 23 bp, Homo sapiens
  modified_base (21)..(21): a, c, t, g, unknown or other
ggcactgcgggtggaggcggngg

SEQ ID NO: 77 - DNA, 23 bp, Homo sapiens
  modified_base (21)..(21): a, c, t, g, unknown or other
gacactgaggcaggaggtggngg

SEQ ID NO: 78 - DNA, 23 bp, Homo sapiens
  modified_base (21)..(21): a, c, t, g, unknown or other
ggctctgtggctggaggaggngg

SEQ ID NO: 79 - DNA, 23 bp, Homo sapiens
  modified_base (21)..(21): a, c, t, g, unknown or other
ggcacttcggttgggggtggngg

SEQ ID NO: 80 - DNA, 23 bp, Homo sapiens
  modified_base (21)..(21): a, c, t, g, unknown or other
gggcacgcggctggaggaggngg

SEQ ID NO: 81 - DNA, 23 bp, Homo sapiens
  modified_base (21)..(21): a, c, t, g, unknown or other
gaggctgcggctgggggtggngg

SEQ ID NO: 82 - DNA, 23 bp, Homo sapiens
  modified_base (21)..(21): a, c, t, g, unknown or other
agcactgtggctgggggaggngg

SEQ ID NO: 83 - DNA, 23 bp, Homo sapiens
  modified_base (21)..(21): a, c, t, g, unknown or other
ggcactgagactgggggtggngg

SEQ ID NO: 84 - DNA, 23 bp, Homo sapiens
  modified_base (21)..(21): a, c, t, g, unknown or other
agcactgcagctgggagtggngg

SEQ ID NO: 85 - DNA, 23 bp, Homo sapiens
  modified_base (21)..(21): a, c, t, g, unknown or other
tgcactgtggctggagatggngg

SEQ ID NO: 86 - DNA, 23 bp, Homo sapiens
  modified_base (21)..(21): a, c, t, g, unknown or other
ggcactggggctgggggaggngg

SEQ ID NO: 87 - DNA, 23 bp, Homo sapiens
  modified_base (21)..(21): a, c, t, g, unknown or other
gacaccgtgactggaggtggngg

SEQ ID NO: 88 - DNA, 23 bp, Homo sapiens
  modified_base (21)..(21): a, c, t, g, unknown or other
cccactggggctggaggtggngg

SEQ ID NO: 89 - DNA, 23 bp, Homo sapiens
  modified_base (21)..(21): a, c, t, g, unknown or other
ggcactgctgctagaggtgcngg

SEQ ID NO: 90 - DNA, 23 bp, Homo sapiens
  modified_base (21)..(21): a, c, t, g, unknown or other
ggctccgcagctggaggtggngg

SEQ ID NO: 91 - DNA, 23 bp, Homo sapiens
  modified_base (21)..(21): a, c, t, g, unknown or other
ggcacggcggctggggtgggngt

SEQ ID NO: 92 - DNA, 23 bp, Homo sapiens
  modified_base (21)..(21): a, c, t, g, unknown or other
gggactgcggctggaggtggnaa

SEQ ID NO: 93 - DNA, 23 bp, Homo sapiens
  modified_base (21)..(21): a, c, t, g, unknown or other
gccactggggctgggggtggngg

SEQ ID NO: 94 - DNA, 23 bp, Homo sapiens
  modified_base (21)..(21): a, c, t, g, unknown or other
tctactgaggctggaggtggngg

SEQ ID NO: 95 - DNA, 23 bp, Homo sapiens
  modified_base (21)..(21): a, c, t, g, unknown or other
ttgcatgcggctggaagtggngg

SEQ ID NO: 96 - DNA, 23 bp, Homo sapiens
  modified_base (21)..(21): a, c, t, g, unknown or other
agcacagcggctggagaaggnag

SEQ ID NO: 97 - DNA, 23 bp, Homo sapiens
  modified_base (21)..(21): a, c, t, g, unknown or other
ggcacagcagctggaggtgcngg

SEQ ID NO: 98 - DNA, 23 bp, Homo sapiens
  modified_base (21)..(21): a, c, t, g, unknown or other
gaccccctccaccccgcctcngg

SEQ ID NO: 99 - DNA, 23 bp, Homo sapiens
  modified_base (21)..(21): a, c, t, g, unknown or other
attcccccccaccccgcctcngg

SEQ ID NO: 100 - DNA, 23 bp, Homo sapiens
  modified_base (21)..(21): a, c, t, g, unknown or other
gcccccacccaccccgcctcngg

SEQ ID NO: 101 - DNA, 23 bp, Homo sapiens
  modified_base (21)..(21): a, c, t, g, unknown or other
ctacccctccaccccgcctcngg

SEQ ID NO: 102 - DNA, 23 bp, Homo sapiens
  modified_base (21)..(21): a, c, t, g, unknown or other
gggcccctccaccccgcctcngg

SEQ ID NO: 103 - DNA, 23 bp, Homo sapiens
  modified_base (21)..(21): a, c, t, g, unknown or other
ctgcccccccaccccgccacngg

SEQ ID NO: 104 - DNA, 23 bp, Homo sapiens
  modified_base (21)..(21): a, c, t, g, unknown or other
acacccccccaccccgcctcngg

SEQ ID NO: 105 - DNA, 23 bp, Homo sapiens
  modified_base (21)..(21): a, c, t, g, unknown or other
gcttccctccaccccgcatcngg

SEQ ID NO: 106 - DNA, 23 bp, Homo sapiens
  modified_base (21)..(21): a, c, t, g, unknown or other
ctccccacccaccccgcctcngg

SEQ ID NO: 107 - DNA, 23 bp, Homo sapiens
  modified_base (21)..(21): a, c, t, g, unknown or other
taccccccacaccccgcctcngg

SEQ ID NO: 108 - DNA, 23 bp, Homo sapiens
  modified_base (21)..(21): a, c, t, g, unknown or other
cgccctccccaccccgcctcngg

SEQ ID NO: 109 - DNA, 23 bp, Homo sapiens
  modified_base (21)..(21): a, c, t, g, unknown or other
tgcccctcccaccccgcctcngg

SEQ ID NO: 110 - DNA, 23 bp, Homo sapiens
  modified_base (21)..(21): a, c, t, g, unknown or other
gaccccctccaccccgcctcngg

SEQ ID NO: 111 - DNA, 23 bp, Homo sapiens
  modified_base (21)..(21): a, c, t, g, unknown or other
cctcccccacaccccgcatcngg

SEQ ID NO: 112 - DNA, 23 bp, Homo sapiens
  modified_base (21)..(21): a, c, t, g, unknown or other
tgccccccccaccccacctcngg

SEQ ID NO: 113 - DNA, 23 bp, Homo sapiens
  modified_base (21)..(21): a, c, t, g, unknown or other
cccccccccccccccgcctcngg

SEQ ID NO: 114 - DNA, 23 bp, Homo sapiens
  modified_base (21)..(21): a, c, t, g, unknown or other
cataccccccaccccgccccngg

SEQ ID NO: 115 - DNA, 23 bp, Homo sapiens
  modified_base (21)..(21): a, c, t, g, unknown or other
ccccacccccaccccgcctcngg

SEQ ID NO: 116 - DNA, 23 bp, Homo sapiens
  modified_base (21)..(21): a, c, t, g, unknown or other
gaccccccccaccccgccccngg

SEQ ID NO: 117 - DNA, 23 bp, Homo sapiens
  modified_base (21)..(21): a, c, t, g, unknown or other
gaccctgtccaccccacctcngg

SEQ ID NO: 118 - DNA, 23 bp, Homo sapiens
  modified_base (21)..(21): a, c, t, g, unknown or other
aggcccccacaccccgcctcngg

SEQ ID NO: 119 - DNA, 23 bp, Homo sapiens
  modified_base (21)..(21): a, c, t, g, unknown or other
gtccccctcctccccacctcngg

SEQ ID NO: 120 - DNA, 23 bp, Homo sapiens
  modified_base (21)..(21): a, c, t, g, unknown or other
cagtccccccaccccacctcngg

SEQ ID NO: 121 - DNA, 23 bp, Homo sapiens
  modified_base (21)..(21): a, c, t, g, unknown or other
ccgcccctccaccccgccacngg

SEQ ID NO: 122 - DNA, 21 bp, Homo sapiens
  modified_base (21)..(21): a, c, t, g, unknown or other
ctcaccccccaccccacctcn

SEQ ID NO: 123 - DNA, 23 bp, Homo sapiens
  modified_base (21)..(21): a, c, t, g, unknown or other
agcccccacctccccgcctcngg

SEQ ID NO: 124 - DNA, 23 bp, Homo sapiens
  modified_base (21)..(21): a, c, t, g, unknown or other
ccacccccccaccccgccccngg

SEQ ID NO: 125 - DNA, 23 bp, Homo sapiens
  modified_base (21)..(21): a, c, t, g, unknown or other
aggcccccccgccccgcctcngg

SEQ ID NO: 126 - DNA, 23 bp, Homo sapiens
  modified_base (21)..(21): a, c, t, g, unknown or other
cattccccccaccccacctcngg

SEQ ID NO: 127 - DNA, 23 bp, Homo sapiens
  modified_base (21)..(21): a, c, t, g, unknown or other
tgccccccccaccccgccccngg

SEQ ID NO: 128 - DNA, 23 bp, Homo sapiens
  modified_base (21)..(21): a, c, t, g, unknown or other
gacccctcccaccccgactcngg

SEQ ID NO: 129 - DNA, 23 bp, Homo sapiens
  modified_base (21)..(21): a, c, t, g, unknown or other
tcccccccacccccgacctcngg

SEQ ID NO: 130 - DNA, 23 bp, Homo sapiens
  modified_base (21)..(21): a, c, t, g, unknown or other
gccccccaccaccccacctcngg

SEQ ID NO: 131 - DNA, 23 bp, Homo sapiens
  modified_base (21)..(21): a, c, t, g, unknown or other
aacctcccccaccccaccccngg

SEQ ID NO: 132 - DNA, 23 bp, Homo sapiens
  modified_base (21)..(21): a, c, t, g, unknown or other
cactccccccaccccgccccngg

SEQ ID NO: 133 - DNA, 23 bp, Homo sapiens
  modified_base (21)..(21): a, c, t, g, unknown or other
accccccccccccccgccccngg

SEQ ID NO: 134 - DNA, 23 bp, Homo sapiens
  modified_base (21)..(21): a, c, t, g, unknown or other
aacccccaccacccacctccngg

SEQ ID NO: 135 - DNA, 23 bp, Homo sapiens
  modified_base (21)..(21): a, c, t, g, unknown or other
gacccctccctccccacctcngg

SEQ ID NO: 136 - DNA, 23 bp, Homo sapiens
  modified_base (21)..(21): a, c, t, g, unknown or other
caacccccccaccccgcttcngg

SEQ ID NO: 137 - DNA, 23 bp, Homo sapiens
  modified_base (21)..(21): a, c, t, g, unknown or other
gacccctaccaccccatctcngg

SEQ ID NO: 138 - DNA, 23 bp, Homo sapiens
  modified_base (21)..(21): a, c, t, g, unknown or other
ttctccctcctccccgcctcngg

SEQ ID NO: 139 - DNA, 23 bp, Homo sapiens
  modified_base (21)..(21): a, c, t, g, unknown or other
taccccccccaccccgccacngg

SEQ ID NO: 140 - DNA, 23 bp, Homo sapiens
  modified_base (21)..(21): a, c, t, g, unknown or other
tccaccccccaccccgccccngg

SEQ ID NO: 141 - DNA, 23 bp, Homo sapiens
  modified_base (21)..(21): a, c, t, g, unknown or other
gacccctcacaccccgccccngg

SEQ ID NO: 142 - DNA, 23 bp, Homo sapiens
  modified_base (21)..(21): a, c, t, g, unknown or other
ctcccccgccaccccgccccngg

SEQ ID NO: 143 - DNA, 23 bp, Homo sapiens
  modified_base (21)..(21): a, c, t, g, unknown or other
ccgggcccccaccccacctcngg

SEQ ID NO: 144 - DNA, 23 bp, Homo sapiens
  modified_base (21)..(21): a, c, t, g, unknown or other
acccccccccgccccgccccngg

SEQ ID NO: 145 - DNA, 23 bp, Homo sapiens
  modified_base (21)..(21): a, c, t, g, unknown or other
gagctcccccaccccgccccngg

SEQ ID NO: 146 - DNA, 23 bp, Homo sapiens
  modified_base (21)..(21): a, c, t, g, unknown or other
gactccctccgccccgcttcnag

SEQ ID NO: 147 - DNA, 23 bp, Homo sapiens
  modified_base (21)..(21): a, c, t, g, unknown or other
ctacccccactccccgcctcngg

SEQ ID NO: 148 - DNA, 23 bp, Homo sapiens
  modified_base (21)..(21): a, c, t, g, unknown or other
gacaccttccaccccgtctcngg

SEQ ID NO: 149 - DNA, 23 bp, Homo sapiens
  modified_base (21)..(21): a, c, t, g, unknown or other
gaccccacctaccccacctcngg

SEQ ID NO: 150 - DNA, 23 bp, Homo sapiens
  modified_base (21)..(21): a, c, t, g, unknown or other
gacacaccccaccccacctcngg

SEQ ID NO: 151 - DNA, 23 bp, Homo sapiens
  modified_base (21)..(21): a, c, t, g, unknown or other
ccccccccccaccccgccccngg

SEQ ID NO: 152 - DNA, 23 bp, Homo sapiens
  modified_base (21)..(21): a, c, t, g, unknown or other
actcccctccaccccggctcngg

SEQ ID NO: 153 - DNA, 23 bp, Homo sapiens
  modified_base (21)..(21): a, c, t, g, unknown or other
cctcacccccaccccacctcngg

SEQ ID NO: 154 - DNA, 23 bp, Homo sapiens
  modified_base (21)..(21): a, c, t, g, unknown or other
tccgccccccaccccacctcngg

SEQ ID NO: 155 - DNA, 23 bp, Homo sapiens
  modified_base (21)..(21): a, c, t, g, unknown or other
gtcctccaccaccccgcctcngg

SEQ ID NO: 156 - DNA, 23 bp, Homo sapiens
  modified_base (21)..(21): a, c, t, g, unknown or other
gtcccctcccaccccgcctcnag

SEQ ID NO: 157 - DNA, 23 bp, Homo sapiens
  modified_base (21)..(21): a, c, t, g, unknown or other
cccccccccccccccgccccngg

SEQ ID NO: 158 - DNA, 23 bp, Homo sapiens
  modified_base (21)..(21): a, c, t, g, unknown or other
cttcccccacaccccgccccngg

SEQ ID NO: 159 - DNA, 23 bp, Homo sapiens
  modified_base (21)..(21): a, c, t, g, unknown or other
tgcaccccccaccccgccccngg

SEQ ID NO: 160 - DNA, 23 bp, Homo sapiens
  modified_base (21)..(21): a, c, t, g, unknown or other
tcctcccccccacccgcctcngg

SEQ ID NO: 161 - DNA, 23 bp, Homo sapiens
  modified_base (21)..(21): a, c, t, g, unknown or other
tctcccctccaccccgccctngg

SEQ ID NO: 162 - DNA, 23 bp, Homo sapiens
  modified_base (21)..(21): a, c, t, g, unknown or other
tccccccccccccccaccccngg

SEQ ID NO: 163 - DNA, 23 bp, Homo sapiens
  modified_base (21)..(21): a, c, t, g, unknown or other
ccccacccccaccccatctcngg

SEQ ID NO: 164 - DNA, 23 bp, Homo sapiens
  modified_base (21)..(21): a, c, t, g, unknown or other
ctccctccccaccccacctcngg

SEQ ID NO: 165 - DNA, 23 bp, Homo sapiens
  modified_base (21)..(21): a, c, t, g, unknown or other
ccccacccccaccccgccccngg

SEQ ID NO: 166 - DNA, 23 bp, Homo sapiens
  modified_base (21)..(21): a, c, t, g, unknown or other
gcacccccccaccccgctccngc

SEQ ID NO: 167 - DNA, 23 bp, Homo sapiens
  modified_base (21)..(21): a, c, t, g, unknown or other
aacaccccccaccccaccccngg

SEQ ID NO: 168 - DNA, 23 bp, Homo sapiens
  modified_base (21)..(21): a, c, t, g, unknown or other
aagaccccccaccccgccccngg

SEQ ID NO: 169 - DNA, 23 bp, Homo sapiens
  modified_base (21)..(21): a, c, t, g, unknown or other
ccacccctcccacccgcctcngg

SEQ ID NO: 170 - DNA, 23 bp, Homo sapiens
  modified_base (21)..(21): a, c, t, g, unknown or other
cactccccacaccccacctcngg

SEQ ID NO: 171 - DNA, 23 bp, Homo sapiens
  modified_base (21)..(21): a, c, t, g, unknown or other
tccaccccccacccagcctcngg

SEQ ID NO: 172 - DNA, 23 bp, Homo sapiens
  modified_base (21)..(21): a, c, t, g, unknown or other
ctcccccccctccccgcctcngg

SEQ ID NO: 173 - DNA, 23 bp, Homo sapiens
  modified_base (21)..(21): a, c, t, g, unknown or other
accccaccccaccccgtctcngg

SEQ ID NO: 174 - DNA, 23 bp, Homo sapiens
  modified_base (21)..(21): a, c, t, g, unknown or other
gcctccccccacccagcctcngg

SEQ ID NO: 175 - DNA, 20 bp, Homo sapiens
gctattctcgcagctcacca

SEQ ID NO: 176 - DNA, 20 bp, Homo sapiens
ggcactgcggctggaggtgg

SEQ ID NO: 177 - DNA, 20 bp, Homo sapiens
gaccccctccaccccgcctc

SEQ ID NO: 178 - DNA, 23 bp, Homo sapiens
  modified_base (21)..(21): a, c, t, g, unknown or other
gctgcagaagggattccatgngg

SEQ ID NO: 179 - DNA, 23 bp, Homo sapiens
  modified_base (21)..(21): a, c, t, g, unknown or other
gctgcagaagggattccaagngg

SEQ ID NO: 180 - DNA, 23 bp, Homo sapiens
  modified_base (21)..(21): a, c, t, g, unknown or other
gctgcagaagggattccatgngg

SEQ ID NO: 181 - DNA, 23 bp, Homo sapiens
  modified_base (21)..(21): a, c, t, g, unknown or other
actgcaaaatggattccatgngg

SEQ ID NO: 182 - DNA, 23 bp, Homo sapiens
  modified_base (21)..(21): a, c, t, g, unknown or other
gctgcaaaaaggattccaggngg

SEQ ID NO: 183 - DNA, 23 bp, Homo sapiens
  modified_base (21)..(21): a, c, t, g, unknown or other
gctgctgaacagattccatgngg

SEQ ID NO: 184 - DNA, 23 bp, Homo sapiens
  modified_base (21)..(21): a, c, t, g, unknown or other
gacgcagaagggactccatgngg

SEQ ID NO: 185 - DNA, 23 bp, Homo sapiens
  modified_base (21)..(21): a, c, t, g, unknown or other
gctgcagtagggactctatgngg

SEQ ID NO: 186 - DNA, 23 bp, Homo sapiens
  modified_base (21)..(21): a, c, t, g, unknown or other
ggtacagaagggcttccatgngg

(19) United States
(12) Patent Application Publication
(10) Pub. No.: US 2022/0411790 A1
(43) Pub. Date: Dec. 29, 2022

(54) A CRISPR-CAS9 PLATFORM WITH AN INTRINSIC OFF-SWITCH AND ENHANCED SPECIFICITY

(71) Applicant: The Johns Hopkins University, Baltimore, MD (US)

(72) Inventors: Roger Zou, Baltimore, MD (US); Yang Liu, Baltimore, MD (US); Taekjip Ha, Baltimore, MD (US)

(21) Appl. No.: 17/770,531

(22) PCT Filed: Oct. 25, 2020
(86) PCT No.: PCT/US20/57255

§ 371 Date: Apr. 20, 2022

Related U.S. Application Data
(60) Provisional application No. 62/926,408, filed on Oct. 25, 2019.

(51) Int. Cl. C12N 15/11; C12N 9/22

(52) U.S. Cl. CPC C12N 15/11; C12N 9/22; C12N 2310/20

(57) ABSTRACT

A gene-editing system includes an engineered photocleavable guide RNA to endow Cas9 nuclease and base editing activities with a built-in mechanism for fast, light-mediated deactivation. In methods of use, the system retains high editing efficiency, natively improves specificity, offers precise spatial and temporal control, improves base editing purity through early deactivation.

### RELATED APPLICATIONS

This application claims the benefit of priority under 35 U.S.C. § 119(e) to U.S. Provisional Application No. 62/926,408 filed Oct. 25, 2019, the entire contents of which is incorporated herein by reference in its entirety.

### STATEMENT REGARDING FEDERALLY SPONSORED RESEARCH

This invention was made with government support under grant numbers 122569 and 1430124 awarded by the National Institutes of Health and the National Science Foundation. The government has certain rights in this invention.

### FIELD OF THE INVENTION

Engineered guide RNAs (gRNAs) for use with gene editing agents, in particular, CRISPR/Cas9, enhance the targeting specificity of Cas9 while enabling an intrinsic light-triggered deactivation of the gene-editing agent.

### BACKGROUND

CRISPRs (clustered regularly interspaced short palindromic repeats) are DNA loci containing short repetitions of base sequences. Each repetition is followed by short segments of “spacer DNA” from previous exposures to a virus. CRISPRs are often associated with Cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and cut these exogenous genetic elements in a manner analogous to RNAi in eukaryotic organisms.

In a CRISPR/Cas9 system, gene editing complexes are assembled. Each complex includes a Cas9 nuclease and a guide RNA (gRNA) complementary to a target sequence in a targeted DNA. The gRNA directs the Cas9 nuclease to engage and cleave the targeted DNA at or near the target sequence. The cleavage produces a blunt double stranded break that, without further intervention, triggers repair enzymes to rejoin or replace DNA sequences at or near the cleavage site. These repairs are usually defective, resulting in one or more mutations into the target DNA, such as nucleotide substitutions, insertions, and deletions. The mutations can included the excision of long stretches of DNA, especially when multiple target sites are cleaved simultaneously.

### SUMMARY

Cas9 is an endonuclease which damages DNA, so the ability to deactivate Cas9 activity on demand is desired; prolonged activity inside cells increases the risk of deleterious side-effects such as off-target editing, genotoxicity and chromosomal translocations.

Accordingly, embodiments of the invention are directed to producing gRNA molecules which greatly enhance the targeting specificity of Cas9 while enabling an intrinsic light-triggered off-switch.

In certain embodiments, a synthetic guide RNA (gRNA) comprises a CRISPR RNA (crRNA), and a trans-activating small RNA (tracrRNA) wherein the crRNA comprises at least one photocleavable linker molecule. In certain embodiments, the at least one photocleavable linker molecule is located between 10 to 20 nucleotides distal to a protospacer adjacent motif (PAM).

In certain embodiments, a synthetic CRISPR RNA (crRNA) oligonucleotide comprises at least one photocleavable linker molecule.

In certain embodiments, a composition comprises an engineered nucleic acid sequence encoding: a clustered regularly interspaced short palindromic repeats (CRISPR)-associated endonuclease, a Cas peptide and at least one guide RNA comprising at least one photocleavable linker molecule. In certain embodiments, the at least one photocleavable linker molecule is located between 10 to 20 nucleotides distal to a protospacer adjacent motif (PAM). In certain embodiments, the engineered nucleic acid sequence further comprises a sequence encoding a transactivating small RNA (tracrRNA). In certain embodiments, the composition comprises at least two or more gRNAs.

In certain embodiments, the composition comprises one or more engineered nucleic acids, where the one or more engineered nucleic acids encode multiple guide nucleic acids, wherein each guide nucleic acid comprises a nucleotide sequence substantially complementary to different target sequences in a genome.

In certain embodiments, an expression vector encoding a clustered regularly interspaced short palindromic repeats (CRISPR)-associated endonuclease, a Cas peptide and at least one guide RNA comprising at least one photocleavable linker molecule. The expression vector may be, without limitation, a lentiviral vector, an adenoviral vector, and an adeno-associated virus vector.

In certain embodiments, a method of deactivating a gene editing agent comprises contacting a cell with a composition comprising a gene editing agent, wherein the gene-editing agent comprises a clustered regularly interspaced short palindromic repeats (CRISPR)-associated endonuclease, a Cas peptide and at least one guide RNA (gRNA) comprising at least one photocleavable linker molecule; subjecting the cell to an electromagnetic radiation, thereby cleaving the at least one gRNA and deactivating the gene-editing agent. In certain embodiments, the electromagnetic radiation comprises a wavelength of between about 190 to about 2400 nm. In certain embodiments, the electromagnetic radiation has a wavelength of about 365 nm. In certain embodiments, the at least one photocleavable linker molecule is located between 10 to 20 nucleotides distal to a protospacer adjacent motif (PAM). In certain embodiments, the photocleavable linker molecule is positioned at 15 nucleotides distal to the PAM, wherein cleavage truncates at a region of target complementarity to 14 nucleotide, rendering Cas9 cleavage-incompetent.

In certain embodiments, the at least one photocleavable linker molecule is capable of reacting with phosphoryl, carboxyl, carbonyl, thiol and amine functionalities. In certain embodiments, the photocleavable linker molecule comprises: 2-nitrobenzyl moieties, alpha-substituted 2-nitrobenzyl moieties, 3,5-dimethoxybenzyl moieties, thiohydroxamic acid, 7-nitroindoline moieties, 9-phenylxanthyl moieties, benzoin moieties, hydroxyphenacyl moieties, or combinations thereof. In certain embodiments, the photocleavable linker is a photocleavable 2-nitrobenzyl linker (PC-Linker).

In certain embodiments, the Cas peptide is Cas9 or a variant thereof. In certain embodiments, the Cas9 variant comprises one or more point mutations, relative to wildtype Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In certain embodiments, the Cas peptide is Cpf1 or a variant thereof.

In certain embodiments, the engineered nucleic acid encoding the Cas peptide is optimized for expression in a human cell.

In certain embodiments, a kit comprises a guide RNA (gRNA), a synthetic CRISPR RNA (crRNA), a composition comprising an engineered nucleic acid sequence encoding for a gRNA, crRNA, tracrRNA or combinations thereof.

In certain embodiments, a nucleic acid sequence comprises a sequence having at least about 70% (such as at least about 75%, 80%, 85%, 90%, 91%, 92%, 93%, 94%, 95%, 96%, 97%, 98%, 99%, or greater) sequence identity to any one of SEQ ID NOS: 1-59.

In certain embodiments, a nucleic acid sequence comprises any one or more of SEQ ID NOS: 1 to 59.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. It is also to be understood that the terminology used herein is for the purpose of describing particular embodiments only, and is not intended to be limiting. In describing and claiming the present invention, the following terminology will be used.

All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

As used herein, the term “expression” as used herein is defined as the transcription and/or translation of a particular nucleotide sequence driven by its promoter.

As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the term “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties. In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

As used herein, the terms “nucleic acid sequence”, “polynucleotide,” are used interchangeably throughout the specification and include complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

As used herein, the term “photocleavable linker” refers to any chemical group that attaches or operably links two nucleotides in an oligonucleotide or polynucleotides. The present invention contemplates photocleavable linkers including, but not limited to, 2-nitrobenzyl moieties, alpha-substituted 2-nitrobenzyl moieties, 3,5-dimethoxybenzyl moieties, thiohydroxamic acid, 7-nitroindoline moieties, 9-phenylxanthyl moieties, benzoin moieties, hydroxyphenacyl moieties, N-hydroxysuccinimidyl-4-azidosalicyclic acid (NHS-ASA), a protective group selected from the group consisting of 9-fluorenylmethoxycarbonyl (Fmoc), 2-(4biphenyl)propyl(2)oxycarbonyl (Bpoc), and derivatives thereof. The present invention also contemplates photocleavable linkers comprising 2-nitrobenzyl moieties and “cross-linker arms” (or “spacer arms”). Examples of such “crosslinker arms” include, but are not limited to, alkyl chains or repeat units of caproyl moieties linked via amide linkages.

As used herein, the term “polynucleotide” is a chain of nucleotides, also known as a “nucleic acid”. As used herein polynucleotides include, but are not limited to, all nucleic acid sequences which are obtained by any means available in the art, and include both naturally occurring and synthetic nucleic acids.

As used herein, the term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “constitutive” promoter is a nucleotide sequence which, when operably linked with a polynucleotide which encodes or specifies a gene product, causes the gene product to be produced in a cell under most or all physiological conditions of the cell.

As used herein, the term “inducible” promoter is a nucleotide sequence which, when operably linked with a polynucleotide which encodes or specifies a gene product, causes the gene product to be produced in a cell substantially only when an inducer which corresponds to the promoter is present in the cell.

As used herein, the term “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

As used herein, the term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

The term “polynucleotide” is a chain of nucleotides, also known as a “nucleic acid”. As used herein polynucleotides include, but are not limited to, all nucleic acid sequences which are obtained by any means available in the art, and include both naturally occurring and synthetic nucleic acids.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

As used herein, the term “variant”, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

As used herein, the term “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1I are a series of schematic representations and graphs demonstrating that photocleavable crRNA retains high editing efficiency with improved targeting specificity. FIG. 1A: Structure of photocleavable (PC) 2-nitrobenzyl linker incorporated into RNA. FIG. 1B: Schematic of Cas9 deactivation by photocleavage of crRNA. FIG. 1C: in vitro cleavage gel of a PCR amplicon containing the PPP1R2 target sequence. n.d. indicates no detectable cleavage. FIG. 1D: In vitro cleavage results for all 4 target sequences: ACTB, HEK site 4, PPP1R2, and VEGFA site 2, demonstrating low basal cleavage activity after light deactivation and uncompromised cleavage activity without light (n=2). FIGS. 1E, 1F: Low basal indel (n=2) and base editing (n=3) activity in cells with light, and high activity without light. FIG. 1G: Cas9 with pcRNA (PC) is more specific than with wtRNA (WT) at all tested off-target loci from targeted deep sequencing (n=2). FIG. 1H: AncBE4max with pcRNA (PC) is more specific than with wtRNA (WT) at the same off-target sites (n=3). FIG. 1I: 97% fewer off-target sites detected with PC compared to WT at HEK site 4 (1 vs 36 off-target sites) using GUIDE-seq. The sole detected off-target site is coincidentally OFF3, consistent with FIG. 1G. FIGS. 1F, 1H: Because multiple Cs are edited per target sequence, the C with the highest editing was chosen for this analysis (C7 for ON target of ACTB; C5 for all ON and OFF targets of HEK site 4; C5 for ON, C5 for OFFS, C7 for OFF23, C3 for OFF24 of VEGFA site 2; C # indicates the cytosine at base #, counting from the PAM-distal end). FIGS. 1D-1H: All error bars represent standard deviations.

FIGS. 2A-2E are a series of graphs and schematic representations demonstrating the temporal and spatial control of deactivation with pcRNA. FIG. 2A: Schematic for temporal control of Cas9/base editor activity with timed light-mediated deactivation. FIG. 2B: Shining light to cells at early time points inhibits endpoint (72 h) Cas9 indels. The x's represent averaged experimental data, error bars represent standard deviations. Lines represent the model predictions after fitting experimental data (n=2). FIGS. 2C, 2D: Shining light to cells at early time points inhibits endpoint AncBE4max cytosine to thymine transitions and indels (n=3). FIG. 2E: Shining light to cells at 3 h (PC 3 hr) enhances endpoint base editing purity compared to no light (PC) or wild type gRNA (WT) (n=3). FIG. 2F: Schematic of spatial control assay using plasmid containing Cas9 cleavage site between mCherry and GFP. Cleavage of plasmid by active Cas9 disrupts both mCherry and GFP expression, but we use GFP as the readout for simplicity. FIG. 2G: Illustration of the masks used when illuminating cells (top row) and the corresponding fluorescence cell imaging for GFP expression 24 h after delivery of reporter plasmid (bottom row). White areas allow free passage of light through mask, which deactivates Cas9 and allows full expression of reporter plasmid in the illuminated cells. Scale bars indicates 1 mm.

FIGS. 3A-3C is a series of blots and schematic representations demonstrating that photocleavable guide RNA (pcRNA) enables light-induced deactivation of Cas9 cleavage in vitro and uncompromised cleavage activity without light. FIG. 3A: Schematic of the native 42 nt pcRNA and truncated 36 nt pcRNA after photocleavage, which both hybridizes with a 67 nt tracrRNA purchased from Integrated DNA Technologies (IDT) (Alt-R® CRISPR-Cas9 tracrRNA). FIG. 3B: In vitro cleavage assay at the ACTB target sequence. All target DNA are the PCR products of genomic DNA from HEK293T cells that contains the target site. Light (30 s, 365 nm) catalyzes photocleavage of the chemical group, leading to a truncated 36 nt pcRNA with 14 bp complementarity to target DNA which renders Cas9 cleavage deficient. The pcRNA band shifts from 42 nt in ‘no light’ sample to 36 nt in ‘light’ sample, indicating complete photocleavage with this dose. Positive contrfl (‘+ctrl’) uses a cleavage-competent 36 nt crRNA purchased from IDT (Alt-R® CRISPR-Cas9 crRNA) hybridized to the same 67 nt tracrRNA. To calculate the cleavage efficiency, the integrated intensity of cleaved bands was divided by that of total DNA as quantified using ImageJ. FIG. 3C: In vitro cleavage assays at three other target sequences: HEK site 4, PPP1R2, and VEGFA site 2.

FIGS. 4A, 4B are blots showing the final eluate of SpCas9 and AncBE4max from His Tag purification after expression in E. coli. FIG. 4A: Gel of eluate for SpCas9 from Ni-NTA agarose beads. E1-E3 were pooled. FIG. 4B: Gel of eluate for AncBE4max. E1-E2 were pooled. Eluted proteins are the expected size for both SpCas9 and AncBE4max.

FIGS. 5A-5F are a series of graphs demonstrating that photocleavable guide RNA (PC) natively exhibits significantly enhanced specificity compared to wild type guide RNA (WT) when used with wild type SpCas9 or AncBE4max cytosine base editor. FIGS. 5A-5D: Percent C to T conversion with AncBE4max at every editable C, at select off-target sites of HEK site 4 (OFF1, OFF3, OFF10) and VEGFA site 2 (OFFS, OFF23, OFF24). pcRNA (PC) greatly reduces off-target editing at every editable C compared to wild type gRNA (WT). C # indicates the cytosine at base #, counting from the PAM-distal end. FIG. 5E: Bar plot comparing the percent of on-target indels divided by the specific off-target indels for all tested off-target sites, between PC and WT. FIG. 5F: Bar plot comparing the percent of on-target C to T transition divided by the specific off-target base transition for all tested off-target sites, between PC and WT. FIGS. 5E, 5F: Because multiple Cs are edited per target sequence, the C with the highest editing was chosen for this analysis (C5 for all ON and OFF targets of HEK site 4; C5 for ON, C5 for OFFS, C7 for OFF23, C3 for OFF24 of VEGFA site 2).

FIGS. 6A and 6B show ordered lists of Cas9 targeting sites from GUIDE-seq of two endogenous target sequences. FIG. 6A: GUIDE-seq at HEK site 4 using wild type guide (WT) versus photocleavable guide (PC) without light, or light 3 h after RNP delivery by electroporation. WT sample had 36 off-target sites, while both PC samples had 1 off-target site detected. FIG. 6B: GUIDE-seq at VEGFA site 2 with the same experimental conditions. WT had 76 off-target sites, while PC no light sample had 4 off-target sites and PC with 3 h of Cas9 activity had 3 off-target sites detected. All samples were harvested at 72 h.

FIGS. 7A and 7B are a series of blots and a graph demonstrating the in vitro kinetic measurements of Cas9 cleavage with photocleavable (PC) or wild type (WT) gRNA. FIG. 7A: Representative gel from in vitro cleavage assays. A 444 bp PCR amplicon from genomic DNA that contains the PPP1R2 target sequence was incubated with SpCas9 in complex with either PC or WT guide RNA at 37° C., and quenched at various time points (30 sec, 1 min, 2 min, 5 min, 10 min, 30 min, 45 min, 1 h). To calculate the cleavage efficiency, the integrated intensity of cleaved bands (218 and 226 bp) was divided by that of total DNA as quantified using ImageJ. FIG. 7B: Summary of gel quantification for each time point (n=2). Cas9 with WT gRNA exhibited significantly faster cleavage kinetics compared to Cas9 with PC gRNA.

FIGS. 8A and B are a series of graphs and a table showing C to T conversion at all editable Cs with different durations of base editor activity. FIG. 8A: C to T conversion at all editable Cs with 0 h, 1 h, 3 h, 6 h, and full 72 h duration of activity for ACTB, HEK site 4, and VEGFA site 2. 0 h indicates RNP inactivated before electroporation; 72 h duration indicates no light. All others indicate light at X h after electroporation; all samples were collected at 72 h. FIG. 8B: Corresponding tabulated reads from targeted deep sequencing. ‘untreated’ indicates wild type HEK293T cells. For each sample (e.g. ACTB—PC no light), each column (G, C, T, A, T, . . . ) represents the expected base call based on the target sequence, and each row (A, C, G, T) represents the actual proportion of base calls with that base. Conditional coloring indicates the range of editing for each base, with white for 0% editing, red for 50% editing, and blue for 100% editing.

FIGS. 9A-9D are a series of graphs demonstrating the rate constants derived from model of temporally-limited genome editing using pcRNA. The full description of the modeling process is found in Example 2. FIG. 9A: kₑ for indels from SpCas9. FIG. 9B: kₑ for both base editing and indels from cytosine base editor AncBE4max. FIG. 9C: k_{d} for both SpCas9 and AncBE4max. FIG. 9: SpCas9-mediated indels at HEK site 4 on-target (ON) and off-target 3 (OFF3). The x's are experimental data, error bars are standard deviations, and lines represent the fitted model (n=2). kₑ for the two curves are plotted as HEK site 4 ON and HEK site 4 OFF3 in FIG. 9A.

FIG. 10 shows the determination of SpCas9 electroporation efficiency from immunofluorescence imaging. SpCas9 in complex with wild type gRNA was electroporated to HEK293T cells and plated on an imaging dish, incubated for 1 h to allow cell adherence, then fixed and stained against Cas9 (magenta) and dsDNA (DAPI; blue) (n=2). Negative control is HEK293T cells without SpCas9 electroporation. Electroporation efficiency was estimated to be 97% from manual counting of Cas9 positive-cells using a hemocytometer.

FIGS. 11A-11J. FIG. 11A: Percent of indels at the on-target and select off-target sites using Cas9 in complex with either pcRNA (red—‘PC’) or wild type guide RNA (blue—‘WT’) targeting HEK site 4 or VEGFA site 2. Error bars represent ±SD across biological replicates (n=3). n.s. indicates not significant; * indicates p<0.05, ** indicates p<0.01, *** indicates p<0.001. FIG. 11B: Same as FIG. 11A, for cytosine base editor AncBE4max. FIG. 11C: Percent of on-target indels divided by the percent of off-target indels for each tested off-target site. Bar plot compare the values between PC and WT used with Cas9. FIG. 11D: same as FIG. 11C, for AncBE4max-mediated cytosine to thymine conversion. FIGS. 11B and 11D: Because multiple cytosines are edited per target sequence, the cytosine with the highest editing was chosen for this analysis (C5 for all ON and OFF targets of HEK site 4; C5 for ON, C5 for OFFS, C7 for OFF23, C3 for OFF24 of VEGFA site 2, where C # is the #th cytosine counting from the PAM distal side). FIG. 11E: Quantification of off-target sites detected using GUIDE-seq for Cas9 in complex with pcRNA (red—‘PC’) or wild type gRNA (blue—‘WT’). Percent reduction in the number of off-target sites from WT to PC for each target sequence is labeled above the bar plot. FIG. 11F: Time-resolved in vitro cleavage efficiencies of the on-target sites for ACTB, HEK site 4, and VEGFA site 2 using Cas9 in complex with either pcRNA (‘PC’) or wild type guide RNA (‘WT’). Error bars represent ±SD across replicates (n=2). FIG. 11G: GUIDE-seq using wild type gRNA (‘WT’) or pcRNA (PC′) targeting FANCF site 2. ‘1 MM’ is a target sequence with 1 mismatch, with GUIDE-seq enrichment using both pcRNA and wild type gRNA. ‘3 MM’ is a target sequence with 3 mismatches, with GUIDE-seq enrichment only using wild type gRNA. The non-mismatched, on-target sequence is annotated with all dots, indicating no mismatches, across the row. FIGS. 11H-11I: Time-resolved in vitro cleavage efficiencies of the on-target site for FANCF site 2 (‘0 MM’) and off-target sites ‘1 MM’ and ‘3 MM’ labeled in FIG. 11G. Error bars represent ±SD across replicates (n=2). FIG. 11J Cleavage at 1 minute from panels H and I are plotted separately, which can be used as an estimate of initial cleavage rates.

FIG. 12. FIGS. 12A-12H. FIG. 12A: Schematic of temporal control of Cas9 or AncBE4max activity with timed light-mediated deactivation—to determine the dose of genome editor required a specific level of editing. FIGS. 12C, 12E, and 12G correspond to Cas9, while FIGS. 12D, 12F, and 12H correspond to AncBE4max. Ef, t, A(t), B(t), refer to quantities defined in the mathematical model (Supplemental Theory I and II). FIG. 12B: Schematic of direct measurement of Cas9 or AncBE4max genome editing at the 15-hour time point—different levels of indels at this relatively early time point reflects differences in genome editing kinetics, which is necessary information for the mathematical model of duration-resolved genome editing (because the required duration is highly dependent on editing kinetics). FIGS. 12C and 12D: Shining light to cells at early time points inhibits endpoint (72 hour) Cas9 indels or AncBE4max base editing (i.e. cytosine to thymine transitions). The circles represent averaged experimental data. For example, the point that align to 12 hours on the x axis indicates an active genome editing duration of 12 hours, followed by deactivation at the 12-hour time point after electroporation. All samples were evaluated for genome editing at 72 hours after electroporation. Lines represent the model predictions after fitting experimental data. Error bars represent ±SD across biological replicates (n=3). FIGS. 12E and 12F: Deactivation at 12 hours followed by indel/base editing measurement at 72 hours (y-axis) is highly correlated with indel/base editing measurements directly at 15 hours (x-axis). Grey points (“model”) correspond to triplicate measurements for ACTB, HEK site 4, and VEGFA site 2, which was used to determine the line of best fit (dotted line). Red points (“prediction”) correspond to triplicate measurements for MYC, a new target sequence to validate the predictive accuracy of our model. FIGS. 12G and 12H: The mathematical model was fit (grey lines) using ACTB, HEK site 4, and VEGFA site 2 data from FIGS. 12C and 12D. For a new target sequence, MYC, given its indel/base editing measurements at 15 hours, indel/base editing measurements at 72 hours after Cas9/AncBE4max deactivation at 12 hours can be predicted using FIGS. 12E and 12F, which can then be used to determine model parameters and predict the full kinetic.

### DETAILED DESCRIPTION

The present invention is directed to the development of a CRISPR/Cas9 system with a built-in off-switch such that Cas9 can be rapidly and irreversibly deactivated with high spatiotemporal precision. The gRNAs can be engineered to work with the various gene editing agents.

Accordingly, in general embodiments, compositions comprise an endonuclease and at least one guide RNA (gRNA) sequence, the guide RNA being complementary to a target nucleic acid sequence in a target gene. In some embodiments, the compositions disclosed herein include nucleic acids encoding an endonuclease, such as Cas9.

Gene Editing Agents: Compositions of the invention include at least one gene editing agent, comprising CRISPR-associated nucleases such as Cas9 and Cpf1 gRNAs, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof. See Schiffer, 2012, J Virol 88(17):8920-8936, incorporated by reference.

In certain embodiments, the compositions include isolated nucleic acid sequences encoding a Cpf1 (CRISPR from Prevotella and Francisella 1) endonuclease, and at least one guide RNA (gRNA), which is complementary to a target DNA sequence in the target gene. The gRNA directs the Cpf1 endonuclease to the target DNA sequence. The resulting double stranded breaks in the DNA inactivate the target gene by causing point mutations, insertions, deletions, or the complete excision of a stretch of DNA including the target gene.

In other embodiments, nuclease systems that can be used include, without limitation, zinc finger nucleases, transcription activator-like effector nucleases (TALENs), meganucleases, or any other system that can be used to degrade or interfere with viral nucleic acid without interfering with the regular function of the host's genetic material.

As referenced above, Argonaute is another potential gene editing system. Argonautes are a family of endonucleases that use 5′ phosphorylated short single-stranded nucleic acids as guides to cleave targets (Swarts, D. C. et al. The evolutionary journey of Argonaute proteins. Nat. Struct. Mol. Biol. 21, 743-753 (2014)). Similar to Cas9, Argonautes have key roles in gene expression repression and defense against foreign nucleic acids (Swarts, D. C. et al. Nat. Struct. Mol. Biol. 21, 743-753 (2014); Makarova, K S., et al. Biol. Direct 4, 29 (2009). Molloy, S. Nat. Rev. Microbiol. 11, 743 (2013); Vogel, J. Science 344, 972-973 (2014). Swarts, D. C. et al. Nature 507, 258-261 (2014); Olovnikov, I., et al. Mol. Cell 51, 594-605 (2013)). However, Argonautes differ from Cas9 in many ways Swarts, D. C. et al. The evolutionary journey of Argonaute proteins. Nat. Struct. Mol. Biol. 21, 743-753 (2014)). Cas9 only exist in prokaryotes, whereas Argonautes are preserved through evolution and exist in virtually all organisms; although most Argonautes associate with single-stranded (ss)RNAs and have a central role in RNA silencing, some Argonautes bind ssDNAs and cleave target DNAs (Swarts, D. C. et al. Nature 507, 258-261 (2014); Swarts, D. C. et al. Nucleic Acids Res. 43, 5120-5129 (2015)). guide RNAs must have a 3′ RNA-RNA hybridization structure for correct Cas9 binding, whereas no specific consensus secondary structure of guides is required for Argonaute binding; whereas Cas9 can only cleave a target upstream of a PAM, there is no specific sequence on targets required for Argonaute. Once Argonaute and guides bind, they affect the physicochemical characteristics of each other and work as a whole with kinetic properties more typical of nucleic-acid-binding proteins (Salomon, W. E., et al. Cell 162, 84-95 (2015)).

CRISPR Associated Endonucleases: The compositions disclosed herein may include nucleic acids encoding a CRISPR-associated endonuclease, such as Cas9. In bacteria, the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR RNA (crRNA). The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM).

In certain embodiments, gRNA molecules are provided which greatly enhance the targeting specificity of Cas9 while enabling an intrinsic light-triggered off-switch. In certain embodiments, the Cas9 is a high-fidelity variant comprising SpCas9-HF, eSpCas9, or HypaCas9. These variants display very low off-target activity due to rationally designed mutations.

In certain embodiments, a guide RNA (gRNA) comprises a CRISPR RNA (crRNA), and a trans-activating small RNA (tracrRNA) wherein the crRNA comprises at least one photocleavable linker molecule. In certain embodiments, the at least one photocleavable linker molecule is located between 10 to 20 nucleotides distal to a protospacer adjacent motif (PAM).

In certain embodiments, a synthetic CRISPR RNA (crRNA) oligonucleotide comprises at least one photocleavable linker molecule.

In certain embodiments, a composition comprises an engineered nucleic acid sequence encoding: a clustered regularly interspaced short palindromic repeats (CRISPR)-associated endonuclease, a Cas peptide and at least one guide RNA comprising at least one photocleavable linker molecule. In certain embodiments, the at least one photocleavable linker molecule is located between 10 to 20 nucleotides distal to a protospacer adjacent motif (PAM). In certain embodiments, the engineered nucleic acid sequence further comprises a sequence encoding a transactivating small RNA (tracrRNA). In certain embodiments, the composition comprises at least two or more gRNAs.

In certain embodiments, the composition comprises one or more engineered nucleic acids, where the one or more engineered nucleic acids encode multiple guide nucleic acids, wherein each guide nucleic acid comprises a nucleotide sequence substantially complementary to different target sequences in a genome.

The crRNA and tracrRNA can be expressed separately or engineered into an artificial fusion small guide RNA (sgRNA) via a synthetic stem loop (AGAAAU) to mimic the natural crRNAItracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately. in certain embodiments, a synthetic or engineered guide RNA (gRNA) comprises a CRISPR RNA (crRNA), and a trans-activating small RNA (tracrRNA) wherein the crRNA comprises at least one photocleavable linker molecule.

| CRISPR Nucleases | Organism Isolated From | PAM Sequence (5′ to 3′) |
|---|---|---|
| SpCas9 | Streptococcus pyogenes | NGG |
| SaCas9 | Staphylococcus aureus | NGRRT orNGRRN |
| NmeCas9 | Neisseria meningitidis | NNNNGATT |
| CjCas9 | Campylobacter jejuni | NNNNRYAC |
| StCas9 | Streptococcus thermophilus | NNAGAAW |
| LbCpf1 | Lachnospiraceae bacterium | TTTV |
| AsCpf1 | Acidaminococcus sp. | TTTV |

As discussed above, Cas9 recognizes a trinucleotide (NGG) protospacer adjacent motif (PAM) to specify the cut site (the 3rd nucleotide from PAM). The invention contemplates other Cas9 variants, which recognize specific PAM sequences. Accordingly, the guide RNAs can be designed to function with any number of Cas9 variants.

The Cas9 nuclease can have a nucleotide sequence identical to the wild type Streptococcus pyogenes sequence. The CRISPR-associated endonuclease may be a sequence from other species, for example other Streptococcus species, such as thermophiles. The Cas9 nuclease sequence can be derived from other species including, but not limited to: Nocardiopsis dassonvillei, Streptomyces pristinaespiralis, Streptomyces viridochromogenes, Streptomyces roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficle, Finegoldia magna, Natranaerobius thermophiles, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Psuedomona aeruginosa, Escherichia coli, or other sequenced bacteria genomes and archaea, or other prokaryotic microogranisms may also be a source of the Cas9 sequence utilized in the embodiments disclosed herein.

The wild type Streptococcus pyogenes Cas9 sequence can be modified. An exemplary and preferred CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from another species, for example other Streptococcus species, such as Thermophilus; Psuedomona aeruginosa, Escherichia coli, or other sequenced bacteria genomes and archaea, or other prokaryotic microogranisms. Alternatively, the wild type Streptococcus pyrogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GI:669193757; KM099232.1 GI:669193761; or KM099233.1 GI:669193765. Alternatively, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI:669193757; KM099232.1 GI:669193761; or KM099233.1 GI:669193765, or Cas9 amino acid sequence of PX330 or PX260 (Addgene, Cambridge, Mass.).

The Cas9 nuclease sequence can be a mutated sequence. For example, the Cas9 nuclease can be mutated in the conserved HNH and RuvC domains, which are involved in strand specific cleavage. In another example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3 S)-2-amino-3-methylpentanoic acid and L-cyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

Cpf1 Endonucleases. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary target sequence (also called protospacer) on the target DNA. Cas9 recognizes a guanine rich trinucleotide (NGG) protospacer adjacent motif (PAM) to specify the cut site (the 3rd nucleotide from PAM). The PAM is adjacent to the 3′ end of the target sequence.

In contrast, Cpf1 recognizes a thymine rich PAM, with a consensus sequence TTN, and that PAM is located at the 5′ end of the target sequence. This gives a CRISPR/Cpf1 system a different repertoire of targets from a CRISPR/Cas9 system, expanding the spectrum of available gene editing targets.

Cpf1-mediated cleavage is favorable for gene editing. As previously stated, Cas9 makes a blunt ended cut in double stranded DNA. This promotes error prone repair and genetic inactivation, but is not favorable for splicing a desired segment of DNA into the cut site. In contrast, Cpf1 makes a staggered cut, leaving a five nucleotide overhang in each DNA strand. This is a favorable cut for incorporating a desired DNA segment, for example by homology-directed repair. Furthermore, the cut site is at the distal end of the target site, far from the region that is most important in determining target specificity, the “seed” sequence near the PAM. With the seed sequence left intact, multiple rounds of editing are possible.

Cpf1 systems are simpler and smaller than Cas9 systems. In order to function, CRISPR/Cas9 system require the processing and assembly of two substituent RNAs, crRNA, which contains the spacer sequence, and tracrRNA. The crRNA and tracrRNA have been engineered into hybrid molecule known as a single small guide RNA (sgRNA), which provides a simpler but still large and complex system. In contrast, all binding and enzymatic functions of Cpf1 require only a single guide RNA, termed gRNA. This simplicity facilitates the design and use of CRISPR/Cpf1 systems.

Cpf1 also lacks one of the two nuclease domains found in Cas9. As a smaller molecule it should be easier to transport, for example, through nuclear pores, to target sites.

In certain embodiments, the Cpf1 comprise Acidaminococcus sp. BV3L6 Cpf1, and Lachnospiraceae bacterium ND2006. These Cpf1 family members have thoroughly characterized, and have been shown to be approximately as effective as Cas9 in editing the DNMT1 gene in human kidney cells (Zetsche B. et al., Cell 163, 1-13 Oct. 22, 2015). Alternatively, the Cpf1 of any species can be utilized, if it can be shown to mediate gRNA guided gene editing in a particular cell type or individual animal. The wild type Acidaminococcus or Lachnospiraceae Cpf1 sequences can be modified to encode biologically active variants of Cpf1, and these variants can have or can include, for example, an amino acid sequence that differs from a wild type Cpf1 by virtue of containing one or more mutations (e.g., an addition, deletion, or substitution mutation or a combination of such mutations). The Cpf1 nucleotide sequence can be modified to encode biologically active variants of Cpf1, and these variants can have or can include, for example, an amino acid sequence that differs from a wild type Cpf1 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cpf1 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cpf1 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cpf1 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3 S)-2-amino-3-methylpentanoic acid and L-cyclopentyl glycine (S)-2-amino-2-cyclopentyl acetic acid. For other examples, one can consult textbooks or the worldwide web (a site is currently maintained by the California Institute of Technology and displays structures of non-natural amino acids that have been successfully incorporated into functional proteins).

For example, the nucleic acid sequence of Cpf1 can be codon optimized for efficient expression in mammalian cells, i.e., “humanized” (Zetsche, et al., 2015).

The Cpf1 nuclease sequence can be mutated to behave as “nickase”, which nicks rather than cleaves DNA, to yield single-stranded breaks. In Cas9, nickase activity is accomplished by mutations in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks (Sander J. D. and Joung L. K., Nature Biotech 32, 347-355 (2014)). The Cpf1's of Acidaminococcus and Lachnospiraceae lack an HNH domain but do include a RuvC domain, so it is likely that a nickase Cpf1 can be created by mutations similar to those employed in Cas9. The biological activity of mutant Cpf1 can be assessed in ways known to one of ordinary skill in the art and includes, without limitation, in vitro cleavage assays or functional assays.

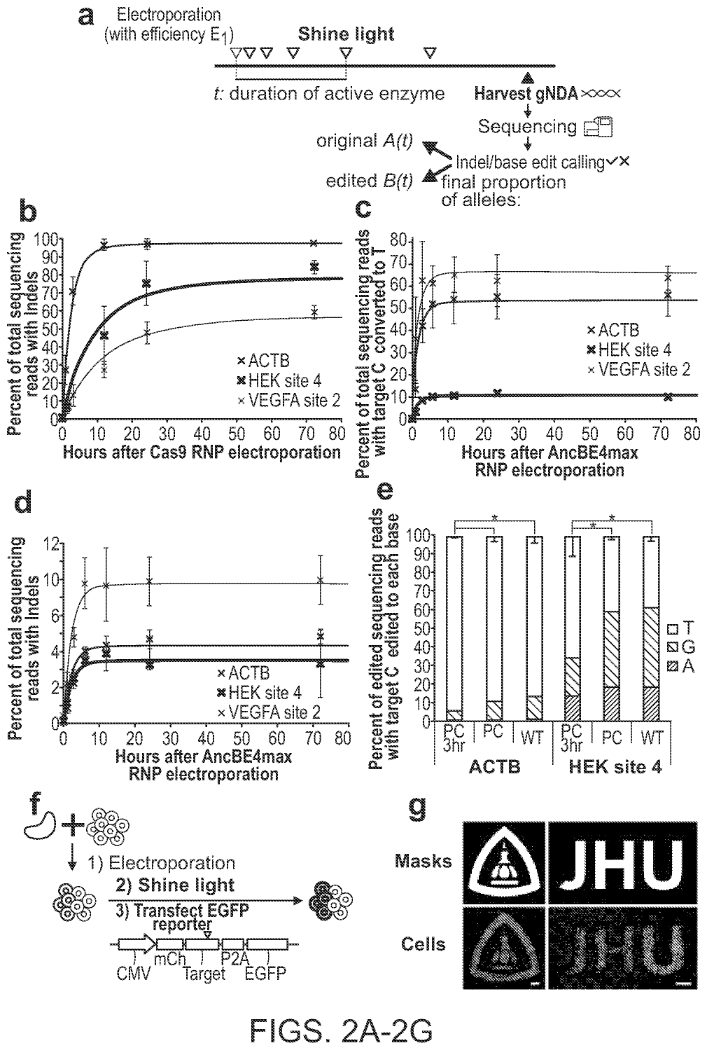

The Cpf1 nuclease sequence can also be mutated to produce a catalytically-deficient Cpf1. A catalytically deficient Cpf1 can be created by suitable mutation of the RuvC domain, as has been accomplished for Cas9 (Gilbert L. A. et al. Cell 154, 442-51 (2013)). A catalytically defective Cpf1 is useful to localize fluorescent labels or regulatory proteins to specific target sites on a DNA molecule. The Cpf1 nuclease sequence can be mutated to produce a Cpf1 with improved targeting efficiency and/or prevents off-targeting of the molecule as compared to the wild-type Cpf1. The Cpf1 molecule can comprise one or more mutations in the Cpf1 nuclease sequence which include, without limitation deletions, substitutions, modified nucleobases, locked nucleic acids, peptide nucleic acids, and the like. The present invention also includes all homologs and orthologues of Cpf1, across all classes of the phyla bacteria and archaea, for example species included in the phylogeny shown in FIG. 2 of Haft D. H., et al. PLoS Comput Biol 1, 0474-0483 (2005). These homologs and orthologues are also included as variant and mutant forms, as previously stated. Cpf1 orthologues, include for example, Cpf1 from Acidaminococcus sp. BV3L6 and Lachnospiraceae bacterium ND 2006 (AsCpf1 and LbCpf1 respectively. These orthologues generally recognize TTTN PAMs that are positioned 5′ to the protospacer.

Guide Nucleic Acid Sequences: Guide RNA sequences according to the present invention can be sense or anti-sense sequences. The specific sequence of the gRNA may vary, but, regardless of the sequence, useful guide RNA sequences will be those that minimize off-target effects while endowing Cas9 with highly efficient nuclease and base editing activities with a built-in mechanism for fast, light-mediated deactivation. This system retains high editing efficiency, natively improves specificity, offers precise spatial and temporal control, and improves base editing purity through early deactivation of the endonuclease activity.

The invention provides for guide RNAs (gRNA) comprising a CRISPR RNA (crRNA), and a trans-activating small RNA (tracrRNA) wherein the crRNA comprises at least one photocleavable linker molecule. In certain embodiments, the at least one photocleavable linker molecule is located between 10 to 20 nucleotides distal to a protospacer adjacent motif (PAM).

The invention also provides for compositions comprising an engineered nucleic acid sequence encoding: a clustered regularly interspaced short palindromic repeats (CRISPR)-associated endonuclease, a Cas peptide and at least one guide RNA comprising at least one photocleavable linker molecule. In certain embodiments, the at least one photocleavable linker molecule is located between 10 to 20 nucleotides distal to a protospacer adjacent motif (PAM). In certain embodiments, the engineered nucleic acid sequence further comprises a sequence encoding a transactivating small RNA (tracrRNA). In certain embodiments, the composition comprises at least two or more gRNAs.

The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs.

Engineered Nucleic Acid Sequences: Methods of producing gRNAs comprising at least one photocleavable linker are described in detail in Example 1 which follows. However, it is contemplated that any standard techniques can be used.

The production of the polynucleotides embodied herein, e.g. vectors, gene-editing agents, isolated nucleic acids, gRNAs, tracrRNA, Cas9, etc., can be produced by any standard technique. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

The nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring portion of a Cas9-encoding DNA (in accordance with, for example, the formula above).

Modified or Mutated Nucleic Acid Sequences: In some embodiments, any of the nucleic acid sequences embodied herein (e.g. addition of photocleavable linkers) may be modified or derived from a native nucleic acid sequence, for example, by introduction of mutations, deletions, substitutions, modification of nucleobases, backbones and the like. The nucleic acid sequences include the vectors, gene-editing agents, isolated nucleic acids, gRNAs, tracrRNA etc. The nucleic acid sequences of the present invention also include variants in which a different base is present at one or more of the nucleotide positions in the compound. For example, if the first nucleotide is an adenosine, variants may be produced which contain thymidine, guanosine or cytidine at this position. This may be done at any of the positions of the isolated nucleic acid sequence. The nucleic acid sequences of the invention may have modifications to the nucleobases or backbones. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH, —N(CH₃)—O—CH₂ [known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequences may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the invention involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651).

In another embodiment, an isolated nucleic acid sequence, e.g. Cas9 or gRNA, comprises combinations of phosphorothioate internucleotide linkages and at least one internucleotide linkage selected from the group consisting of: alkylphosphonate, phosphorodithioate, alkylphosphonothioate, phosphoramidate, carbamate, carbonate, phosphate triester, acetamidate, carboxymethyl ester, and/or combinations thereof. In another embodiment, an isolated nucleic acid sequence optionally comprises at least one modified nucleobase comprising, peptide nucleic acids, locked nucleic acid (LNA) molecules, analogues, derivatives and/or combinations thereof. It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

Certain isolated nucleic acid sequences of this invention are chimeric molecules. “Chimeric molecules” or “chimeras,” in the context of this invention, are isolated nucleic acid sequences which contain two or more chemically distinct regions, each made up of at least one nucleotide. These isolated nucleic acid sequences typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense modulation of gene expression. Consequently, comparable results can often be obtained with shorter isolated nucleic acid sequences when chimeric isolated nucleic acid sequences are used, compared to phosphorothioate deoxyoligonucleotides hybridizing to the same target region.

Chimeric isolated nucleic acid sequences of the invention may be formed as composite structures of two or more oligonucleotides, modified oligonucleotides, oligonucleosides and/or oligonucleotide mimetics as described above. Such; compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In another embodiment, the region of the isolated nucleic acid sequence which is modified comprises at least one nucleotide modified at the 2′ position of the sugar, most preferably a 2′-O-alkyl, 2′-O-alkyl-O-alkyl or 2′-fluoro-modified nucleotide. In another embodiment, the isolated nucleic acid sequences can also be modified to enhance nuclease resistance. Cells contain a variety of exo- and endo-nucleases which can degrade nucleic acids. A number of nucleotide and nucleoside modifications have been shown to make nucleic acid sequence into which they are incorporated more resistant to nuclease digestion than the native oligodeoxynucleotide. Nuclease resistance is routinely measured by incubating isolated nucleic acid sequences with cellular extracts or isolated nuclease solutions and measuring the extent of intact oligonucleotide remaining over time, usually by gel electrophoresis. Isolated nucleic acid sequences which have been modified to enhance their nuclease resistance survive intact for a longer time than unmodified isolated nucleic acid sequences. A variety of oligonucleotide modifications have been demonstrated to enhance or confer nuclease resistance. Isolated nucleic acid sequences can contain at least one phosphorothioate modification. In some cases, oligonucleotide modifications which enhance target binding affinity are also, independently, able to enhance nuclease resistance. Some desirable modifications can be found in De Mesmaeker et al. Acc. Chem. Res. 1995, 28:366-374.

In some embodiments, the RNA molecules e.g. crRNA, tracrRNA, gRNA, are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶Nmethyladenosine; N⁶, N⁶-dimethyladenosine; N⁶,2′-O-trimethyladenosine; 2 methylthio-N⁶Nisopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶ threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1; 2′-O-dimethyl inosine; 2′-O-methyl guanosine; 1-methyl guanosine; N²-methyl guanosine; N², N²-dimethyl guanosine; N², 2′-O-dimethyl guanosine; N², N², 2′-O-trimethyl guanosine; 2′-O-ribosyl guanosine (phosphate); 7-methyl guanosine; N²; 7-dimethyl guanosine; N²; N²; 7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

In other embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher T. (i.e., higher target binding affinity) than 2′-deoxyoligonucleotides against a given target.

In certain embodiments, the modified RNA molecules include at least one photocleavable linker, e.g. 2-nitrobenzyl linker (PC-Linker).

Cleavable Linkers

It is also contemplated that in certain embodiments, the gRNA molecules comprise at least one cleavable linker. Cleavable linkers are known in the art, of which examples include, but are not limited to the ones that are sensitive to an enzyme, pH, temperature, light, shear stress, sonication, a chemical agent (e.g., dithiothreitol), or any combination thereof. In some embodiments, the cleavable linker can be sensitive to light and protein degradation, e.g., by an enzyme.

Cleavable linkers are susceptible to cleavage agents, e.g., hydrolysis, pH, redox potential, and light (e.g., infra-red, and/or UV) or the presence of degradative molecules. Examples of such degradative agents include: redox agents which are selected for particular substrates or which have no substrate specificity, including, e.g., oxidative or reductive enzymes or reductive agents such as mercaptans, present in cells, that can degrade a redox cleavable linker by reduction; esterases; amidases; endosomes or agents that can create an acidic environment, e.g., those that result in a pH of five or lower; enzymes that can hydrolyze or degrade an acid cleavable linker by acting as a general acid, peptidases (which can be substrate specific) and proteases, and phosphatases. In some embodiments, the cleavable linker can be cleavable by a particular enzyme.

In some embodiments, the cleavable, non-hybridizable linker can comprise a photocleavable linker. A photocleavable linker is a linker that can be cleaved by exposure to electromagnetic radiation (e.g., visible light, UV light, infrared, etc.). The wavelength of light necessary to photocleave the linker is dependent upon the structure of the photocleavable linker used. Any art-recognized photocleavable linker can be used for the target probes described herein. Exemplary photocleavable linker include, but are not limited to, chemical molecules containing an o-nitrobenzyl moiety, a p-nitrobenzyl moiety, a m-nitrobenzyl moiety, a nitoindoline moiety, a bromo hydroxycoumarin moiety, a bromo hydroxyquinoline moiety, a hydroxyphenacyl moiety, a dimethozybenzoin moiety, or any combinations thereof.

Additional exemplary photocleavable groups are generally described and reviewed in Pelliccioli et al., Photoremovable protecting groups: reaction mechanisms and applications, Photochem. Photobiol. Sci. 1 441-458 (2002); Goeldner and Givens, Dynamic Studies in Biology, Wiley-VCH, Weinheim (2005); Marriott, Methods in Enzymology, Vol. 291, Academic Press, San Diego (1998); Morrison, Bioorganic Photochemistry, Vol. 2, Wiley, New York (1993); Adams and Tsien, Annu. Rev. Physiol. 55 755-784 (1993); Mayer et al., Biologically Active Molecules with a “Light Switch,” Angew. Chem. Int. Ed. 45 4900-4921 (2006); Pettit et al., Neuron 19 465-471 (1997); Furuta et al., Brominated 7-hydroxycoumarin-4-ylmethyls: Photolabile protecting groups with biologically useful cross-sections for two photon photolysis, Proc. Natl. Acad. Sci. USA 96 1193-1200 (1999); and U.S. Pat. Nos. 5,430,175; 5,635,608; 5,872,243; 5,888,829; 6,043,065; and Zebala, U.S. Patent Application No. 2010/0105120, the disclosures of which are incorporated by reference herein.

In some embodiments, the photocleavable linker can generally be described as a chromophore. Examples of chromophores which are photoresponsive to such wavelengths include, but are not limited to, acridines, nitroaromatics, and arylsulfonamides. The efficiency and wavelength at which the chromophore becomes photoactivated and thus releases the identification nucleotide sequences described herein will vary depending on the particular functional group(s) attached to the chromophore. For example, when using nitroaromatics, such as derivatives of o-nitrobenzylic compounds, the absorption wavelength can be significantly lengthened by addition of methoxy groups.

In some embodiments, the photocleavable linker can comprise a nitro-aromatic compound. Exemplary photocleavable linkers having an ortho-nitro aromatic core scaffold include, but are not limited to, ortho-nitro benzyl (“ONB”), 1-(2-nitrophenyl)ethyl (“NPE”), alpha-carboxy-2-nitrobenzyl (“CNB”), 4,5-dimethoxy-2-nitrobenzyl (“DMNB”), 1-(4,5-dimethoxy-2-nitrophenyl)ethyl (“DMNPE”), 5-carboxymethoxy-2-nitrobenzyl (“CMNB”) and ((5-carboxymethoxy-2-nitrobenzyl)oxy)carbonyl (“CMNCBZ”) photolabile cores. It will be appreciated that the substituents on the aromatic core are selected to tailor the wavelength of absorption, with electron donating groups (e.g., methoxy) generally leading to longer wavelength absorption. For example, nitrobenzyl (“NB”) and nitrophenylethyl (“NPE”) are modified by addition of two methoxy residues into 4,5-dimethoxy-2-nitrobenzyl and 1-(4,5-dimethoxy-2-nitrophenyl)ethyl, respectively, thereby increasing the absorption wavelength range to 340-360 nm.

Further, other ortho-nitro aromatic core scaffolds include those that trap nitroso byproducts in a hetero Diels Alder reaction as generally discussed in Zebala, U.S. Patent Application No. 2010/0105120 and Pirrung et al., J. Org. Chem. 68: 1 138 (2003). The nitrodibenzofurane (“NDBF”) chromophore offers an extinction coefficient significantly higher in the near UV region but it also has a very high quantum yield for the deprotection reaction and it is suitable for two-photon activation (Momotake et al, The nitrodibenzofuran chromophore: a new caging group for ultra-efficient photolysis in living cells, Nat. Methods 3 35-40 (2006)). The NPP group is an alternative introduced by Pfleiderer et al. that yields a less harmful nitrostyryl species (Walbert et al., Photolabile Protecting Groups for Nucleosides: Mechanistic Studies of the 2-(2-Nitrophenyl)ethyl Group, Helv. Chim. Acta 84 1601-1611 (2001)).

In exemplary embodiments involving UV light, the photocleavable linkers can be selected from the group consisting of alpha-carboxy-2-nitrobenzyl (CNB, 260 nm), 1-(2-nitrophenyl)ethyl (NPE, 260 nm), 4,5-dimethoxy-2-nitrobenzyl (DMNB, 355 nm), dimethoxy-2-nitrophenyl)ethyl (DMNPE, 355 nm), (4,5-dimethoxy-2-nitrobenzoxy)carbonyl (NVOC, 355 nm), 5-carboxymethoxy-2-nitrobenzyl (CMNB, 320 nm), ((5-carboxymethoxy-2-nitrobenzyl)oxy)carbonyl (CMNCBZ, 320 nm), desoxybenzoinyl (desyl, 360 nm), and anthraquino-2-ylmethoxycarbonyl (AQMOC, 350 nm).

Other suitable photocleavable linkers are based on the coumarin system, such as BHC (Furuta and Iwamura, Methods Enzymol. 291 50-63 (1998); Furuta et al., Proc. Natl. Acad. Sci. USA 96 1193-1200 (1999); Suzuki et al., Org. Lett. 5:4867 (2003); U.S. Pat. No. 6,472,541, the disclosure of which is incorporated by reference herein). The DMACM linkage photocleaves in nanoseconds (Hagen et al., [7-(Dialkylamino)coumarin-4-yl]methyl-Caged Compounds as Ultrafast and Effective Long-Wavelength Phototriggers of 8-Bromo-Substituted Cyclic Nucleotides, Chem Bio Chem 4 434-442 (2003)) and is cleaved by visible light (U.S. patent application Ser. No. 11/402,715 the disclosure of which is incorporated by reference herein). Coumarin-based photolabile linkages are also available for linking to aldehydes and ketones (Lu et al., Bhc-diol as a photolabile protecting group for aldehydes and ketones, Org. Lett. 5 2119-2122 (2003)). Closely related analogues, such as BHQ, are also suitable (Fedoryak et al., Brominated hydroxyquinoline as a photolabile protecting group with sensitivity to multiphoton excitation, Org. Lett. 4 3419-3422 (2002)). Another suitable photocleavable linker comprises the pHP group (Park and Givens, J. Am. Chem. Soc. 119:2453 (1997), Givens et al., New Phototriggers 9: p-Hydroxyphenacyl as a C-Terminal Photoremovable Protecting Group for Oligopeptides, J. Am. Chem. Soc. 122 2687-2697 (2000); Zhang et al., J. Am. Chem. Soc. 121 5625-5632, (1999); Conrad et al., J. Am. Chem. Soc. 122 9346-9347 (2000); Conrad et al., Org. Lett. 2 1545-1547 (2000)). A ketoprofen derived photolabile linkage is also suitable (Lukeman et al., Carbanion-Mediated Photocages: Rapid and Efficient Photorelease with Aqueous Compatibility, J. Am. Chem. Soc. 127 7698-7699 (2005)).

In some embodiments, a photocleavable linker is one whose covalent attachment to an identification nucleotide sequence and/or target-binding agent is reversed (cleaved) by exposure to light of an appropriate wavelength. In some embodiments, release of the identification nucleotide sequences occurs when the conjugate is subjected to ultraviolet light. For example, photorelease of the identification nucleotide sequences can occur at a wavelength ranging from about 200 to 380 nm (the exact wavelength or wavelength range will depend on the specific photocleavable linker used, and can be, for example, 200, 210, 220, 230, 240, 250, 260, 270, 280, 290, 300, 310, 320, 330, 340, 350, 360, 370, or 380 or some range therebetween). In some embodiments, release of the identification nucleotide sequences occurs when the conjugate is subjected to visible light. For example, photorelease of the identification nucleotide sequences can occur at a wavelength ranging from about 380 to 780 nm (the exact wavelength or wavelength range will depend on the specific photocleavable linker used, and could be, for example, 380, 400, 450, 500, 550, 600, 650, 700, 750, or 780, or some range therebetween). In some embodiments, release of the identification nucleotide sequences occurs when the conjugate is subjected to infrared light. For example, photorelease of the identification nucleotide sequences can occur at a wavelength ranging from about 780 to 1200 nm (the exact wavelength or wavelength range will depend on the specific photocleavable linker used, and could be for example, 780, 800, 850, 900, 950, 1000, 1050, 1100, 1150, or 1200, or some range therebetween).

In some embodiments, the photocleavable linker is a photocleavable bifunctional linker. In some embodiments, the photocleavable linker is a photocleavable multi-functional linker.

In some embodiments where a photocleavable linker is used, the identification nucleotide sequences can be released from the bound target probes by exposing the bound target probes to a light of a specified wavelength. In some embodiments, ultraviolet (UV) light or near UV light can be used to release identification nucleotide sequences from bound target probes. In some embodiments, release of the identification nucleotide sequences can occur at a wavelength ranging from about 200 nm to about 450 nm.

In some embodiments, the cleavable linker is a cleavable, non-hybridizable linker. Exemplary cleavable, non-hybridizable linkers include, but are not limited to, hydrolyzable linkers, redox cleavable linkers (e.g., —S—S— and —C(R)₂—S—S—, wherein R is H or C₁-C₆ alkyl and at least one R is C₁-C₆ alkyl such as CH₃ or CH₂CH₃); phosphate-based cleavable linkers (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(OR)—S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C₁-C₁₀ alkyl); acid cleavable linkers (e.g., hydrazones, esters, and esters of amino acids, —C.═NN— and —OC(O)—); ester-based cleavable linkers (e.g., —C(O)O—); peptide-based cleavable linkers, (e.g., linkers that are cleaved by enzymes such as peptidases and proteases in cells, e.g., —NHCHR^{A}C(O)NHCHR^{B}C(O)—, where RA and RB are the R groups of the two adjacent amino acids), photocleavable linkers and any combinations thereof. A peptide based cleavable linker comprises two or more amino acids. In some embodiments, the peptide-based cleavage linkage comprises the amino acid sequence that is the substrate for a peptidase or a protease. In some embodiments, an acid cleavable linker is cleavable in an acidic environment with a pH of about 6.5 or lower (e.g., about 6.5, 6.0, 5.5, 5.0, or lower), or by agents such as enzymes that can act as a general acid.

In some embodiments, the cleavable, non-hybridizable linker can comprise a disulfide bond, a tetrazine-trans-cyclooctene group, a sulfhydryl group, a nitrobenzyl group, a nitoindoline group, a bromo hydroxycoumarin group, a bromo hydroxyquinoline group, a hydroxyphenacyl group, a dimethozybenzoin group, or any combinations thereof.

Activation agents can be used to activate the components to be conjugated together (e.g., identification nucleotide sequences and/or target-binding molecules). Without limitations, any process and/or reagent known in the art for conjugation activation can be used. Exemplary surface activation method or reagents include, but are not limited to, 1-Ethyl-3-[3-dimethylaminopropyl]carbodiimide hydrochloride (EDC or EDAC), hydroxybenzotriazole (HOBT), N-Hydroxysuccinimide (NHS), 2-(1H-7-Azabenzotriazol-1-yl)-1,1,3,3-tetramethyl uronium hexafluorophosphate methanaminium (HATU), silanization, sulfosuccinimidyl 6-[3′(2-[pyridyldithio)-propionamido] hexanoate (sulfo-LC-SPDP), 2-iminothiolane (Traut's agent), trans-cyclooctene N-hydroxy-succinimidyl ester (TCO-NHS), surface activation through plasma treatment, and the like.

Again, without limitations, any art known reactive group can be used for coupling a photocleavable linker between nucleotides. For example, various surface reactive groups can be used for surface coupling including, but not limited to, alkyl halide, aldehyde, amino, bromo or iodoacetyl, carboxyl, hydroxyl, epoxy, ester, silane, thiol, and the like.

Recombinant Constructs and Delivery Vehicles.

Exemplary expression vectors for inclusion in the pharmaceutical composition include plasmid vectors and lentiviral vectors, but the present invention is not limited to these vectors. A wide variety of host/expression vector combinations may be used to express the nucleic acid sequences described herein. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.). A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAG™ tag (Kodak, New Haven, Conn.) sequences typically are expressed as a fusion with the encoded polypeptide. Such tags can be inserted anywhere within the polypeptide, including at either the carboxyl or amino terminus. The vector can also include origins of replication, scaffold attachment regions (SARs), regulatory regions and the like. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns. The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence. Suitable promoters which may be employed include, but are not limited to, the retroviral LTR; the SV40 promoter; and the human cytomegalovirus (CMV) promoter described in Miller, et al., Biotechniques, Vol. 7, No. 9, 980-990 (1989), or any other promoter (e.g., cellular promoters such as eukaryotic cellular promoters including, but not limited to, the histone, pol III, and β-actin promoters). Other viral promoters which may be employed include, but are not limited to, adenovirus promoters, TK promoters, and B19 parvovirus promoters.

Expression of the/guide nucleic acid sequences may be controlled by any promoter/enhancer element known in the art, but these regulatory elements must be functional in the host selected for expression. Promoters which may be used to control gene expression include, but are not limited to, cytomegalovirus (CMV) promoter (U.S. Pat. Nos. 5,385,839 and 5,168,062), the SV40 early promoter region (Benoist and Chambon, 1981, Nature 290:304-310), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 78:1441-1445, 1981), the regulatory sequences of the metallothionein gene (Brinster et al., Nature 296:39-42, 1982); prokaryotic expression vectors such as the β-lactamase promoter (Villa-Kamaroff, et al., Proc. Natl. Acad. Sci. U.S.A. 75:3727-3731, 1978), or the tac promoter (DeBoer, et al., Proc. Natl. Acad. Sci. U.S.A. 80:21-25, 1983); promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., Cell 38:639-646, 1984; Ornitz et al., Cold Spring Harbor Symp. Quant. Biol. 50:399-409, 1986; MacDonald, Hepatology 7:425-515, 1987); insulin gene control region which is active in pancreatic beta cells (Hanahan, Nature 315:115-122, 1985), immunoglobulin gene control region which is active in lymphoid cells (Grosschedl et al., Cell 38:647-658, 1984; Adames et al., Nature 318:533-538, 1985; Alexander et al., Mol. Cell. Biol. 7:1436-1444, 1987), mouse mammary tumor virus control region which is active in testicular, breast, lymphoid and mast cells (Leder et al., Cell 45:485-495, 1986), albumin gene control region which is active in liver (Pinkert et al., Genes and Devel. 1:268-276, 1987), alpha-fetoprotein gene control region which is active in liver (Krumlauf et al., Mol. Cell. Biol. 5:1639-1648, 1985; Hammer et al., Science 235:53-58, 1987), alpha 1-antitrypsin gene control region which is active in the liver (Kelsey et al., Genes and Devel. 1: 161-171, 1987), beta-globin gene control region which is active in myeloid cells (Mogram et al., Nature 315:338-340, 1985; Kollias et al., Cell 46:89-94, 1986), myelin basic protein gene control region which is active in oligodendrocyte cells in the brain (Readhead et al., Cell 48:703-712, 1987), myosin light chain-2 gene control region which is active in skeletal muscle (Sani, Nature 314:283-286, 1985), and gonadotropic releasing hormone gene control region which is active in the hypothalamus (Mason et al., Science 234:1372-1378, 1986).

In another embodiment the invention comprises an inducible promoter. One such promoter is the tetracycline-controlled transactivator (tTA)-responsive promoter (tet system), a prokaryotic inducible promoter system which has been adapted for use in mammalian cells. The tet system was organized within a retroviral vector so that high levels of constitutively-produced tTA mRNA function not only for production of tTA protein but also the decreased basal expression of the response unit by antisense inhibition. See, Paulus, W. et al., “Self-Contained, Tetracycline-Regulated Retroviral Vector System for Gene Delivery to Mammalian Cells”, J of Virology, January. 1996, Vol. 70, No. 1, pp. 62-67. The selection of a suitable promoter will be apparent to those skilled in the art from the teachings contained herein.

The present invention provides expression vectors for use in expressing the nucleic acid sequences a host cell. Each expression vector includes at least one isolated nucleic acid sequence encoding, for example, Cas9, an endonuclease, at least one (gRNA), and the like. A nucleic acid sequence encoding an endonuclease, and a nucleic acid sequence encoding at least one gRNA, can be included in a single expression vector, or in separate vectors.

In certain embodiments, the vector for expressing the gene editing systems of the invention in mammalian cells is a lentiviral vector, because of its high transduction efficiency and low toxicity. Other suitable expression vectors include, without limitation, plasmids and viral vectors derived from, for example, bacteriophage, baculoviruses, retroviruses. adenoviruses (“Ad”), adeno-associated viruses (AAV), and vesicular stomatitis virus (VSV), and pox viral vectors such as avipox or orthopox vectors. Additional expression vectors also can include derivatives of SV40 and known bacterial plasmids, e.g., E. coli plasmids col E1, pCR1, pBR322, pMal-C2, pET, pGEX, pMB9 and their derivatives; plasmids such as RP4; phage DNAs, e.g., the numerous derivatives of phage 1, e.g., NM989, and other phage DNA, e.g., M13 and filamentous single stranded phage DNA; yeast plasmids such as the 2p, plasmid or derivatives thereof; and vectors derived from combinations of plasmids and phage DNAs, such as plasmids that have been modified to employ phage DNA or other expression control sequences. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.). Suitable promoters and enhancers can be included in the vectors, with the selection being made according to the cell type in which expression is desired, by experimental means well known in the art.

The polynucleotides of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989). Therefore, the present invention encompasses a lentiviral vector composition for expression in a host cell. The composition includes an isolated nucleic acid encoding an endonuclease, and at least one isolated nucleic acid encoding at least one guide gRNA including a spacer sequence that is complementary to a desired target sequence and includes at least one photocleavable linker, with the isolated nucleic acids being included in at least one lentiviral expression vector. The lentiviral expression vector induces the expression of the endonuclease and the at least one gRNA in a host cell.

All of the isolated nucleic acids can be included in a single lentiviral expression vector, or the nucleic acids can be subdivided into any suitable combination of lentiviral vectors. For example, the endonuclease can be incorporated into a first lentiviral expression vector, a first gRNA can be incorporated into a second lentiviral expression vector, and a second gRNA can be incorporated into a third lentiviral expression vector. When multiple expression vectors are used, it is not necessary all of them be lentiviral vectors.

Recombinant constructs are also provided herein and can be used to transform cells.

Several delivery methods may be utilized in conjunction with the molecules embodied herein for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al, J Virol, 72:8463-8471 1998). In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al, Curr Gene Ther, 5:299-310, 2005).

In certain embodiments of the invention, non-viral vectors may be used to effectuate transfection. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat. No. 6,890,554 to Jesse, the contents of each of which are incorporated by reference. Delivery can be to cells (e.g. in vitro or ex vivo administration) or target tissues (e.g. in vivo administration).

Synthetic vectors are typically based on cationic lipids or polymers which can complex with negatively charged nucleic acids to form particles with a diameter in the order of 100 nm. The complex protects nucleic acid from degradation by nuclease. Moreover, cellular and local delivery strategies have to deal with the need for internalization, release, and distribution in the proper subcellular compartment. Systemic delivery strategies encounter additional hurdles, for example, strong interaction of cationic delivery vehicles with blood components, uptake by the reticuloendothelial system, kidney filtration, toxicity and targeting ability of the carriers to the cells of interest. Modifying the surfaces of the cationic non-virals can minimize their interaction with blood components, reduce reticuloendothelial system uptake, decrease their toxicity and increase their binding affinity with the target cells. Binding of plasma proteins (also termed opsonization) is the primary mechanism for RES to recognize the circulating nanoparticles. For example, macrophages, such as the Kupffer cells in the liver, recognize the opsonized nanoparticles via the scavenger receptor.

The nucleic acid sequences of the invention can be delivered by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprises a sequence encoding an endonuclease and/or a guide RNA with a photocleavable linker, as described above.

In some embodiments, delivery of vectors can also be mediated by exosomes. Exosomes are lipid nanovesicles released by many cell types. They mediate intercellular communication by transporting nucleic acids and proteins between cells. Exosomes contain RNAs, miRNAs, and proteins derived from the endocytic pathway. They may be taken up by target cells by endocytosis, fusion, or both. Exosomes can be harnessed to deliver nucleic acids to specific target cells.

The expression constructs of the present invention can also be delivered by means of nanoclews. Nanoclews are a cocoon-like DNA nanocomposites (Sun, et al., J. Am. Chem. Soc. 2014, 136:14722-14725). They can be loaded with nucleic acids for uptake by target cells and release in target cell cytoplasm. Methods for constructing nanoclews, loading them, and designing release molecules can be found in Sun, et al. (Sun W, et al., J. Am. Chem. Soc. 2014, 136:14722-14725; Sun W, et al., Angew. Chem. Int. Ed. 2015: 12029-12033.)

The nucleic acids and vectors may also be applied to a surface of a device (e.g., a catheter) or contained within a pump, patch, or any other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments of the invention, liposomes are used to effectuate transfection into a cell or tissue. The pharmacology of a liposomal formulation of nucleic acid is largely determined by the extent to which the nucleic acid is encapsulated inside the liposome bilayer. Encapsulated nucleic acid is protected from nuclease degradation, while those merely associated with the surface of the liposome is not protected. Encapsulated nucleic acid shares the extended circulation lifetime and biodistribution of the intact liposome, while those that are surface associated adopt the pharmacology of naked nucleic acid once they disassociate from the liposome. Nucleic acids may be entrapped within liposomes with conventional passive loading technologies, such as ethanol drop method (as in SALP), reverse-phase evaporation method, and ethanol dilution method (as in SNALP).

Liposomal delivery systems provide stable formulation, provide improved pharmacokinetics, and a degree of ‘passive’ or ‘physiological’ targeting to tissues. Encapsulation of hydrophilic and hydrophobic materials, such as potential chemotherapy agents, are known. See for example U.S. Pat. No. 5,466,468 to Schneider, which discloses parenterally administrable liposome formulation comprising synthetic lipids; U.S. Pat. No. 5,580,571, to Hostetler et al. which discloses nucleoside analogues conjugated to phospholipids; U.S. Pat. No. 5,626,869 to Nyqvist, which discloses pharmaceutical compositions wherein the pharmaceutically active compound is heparin or a fragment thereof contained in a defined lipid system comprising at least one amphiphatic and polar lipid component and at least one nonpolar lipid component.

Liposomes and polymerosomes can contain a plurality of solutions and compounds. In certain embodiments, the complexes of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21(20):9183-6, incorporated by reference.

In some embodiments of the invention, targeted controlled-release systems responding to the unique environments of tissues and external stimuli are utilized. Gold nanorods have strong absorption bands in the near-infrared region, and the absorbed light energy is then converted into heat by gold nanorods, the so-called “photothermal effect”. Because the near-infrared light can penetrate deeply into tissues, the surface of gold nanorod could be modified with nucleic acids for controlled release. When the modified gold nanorods are irradiated by near-infrared light, nucleic acids are released due to thermo-denaturation induced by the photothermal effect. The amount of nucleic acids released is dependent upon the power and exposure time of light irradiation.

Regardless of whether compositions are administered as nucleic acids or polypeptides, they are formulated in such a way as to promote uptake by the mammalian cell. Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

In other embodiments, the compositions comprise a cell which has been transformed or transfected with one or more Cas9 encoding vectors and gRNAs. In some embodiments, the methods of the invention can be applied ex vivo. That is, a subject's cells can be removed from the body and treated with the compositions in culture to excise, for example, desired nucleic acid sequences e.g. viral infections such as HIV and the treated cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

Transduced cells are prepared for reinfusion according to established methods. After a period of about 2-4 weeks in culture, the cells may number between 1×10⁶ and 1×10¹⁰. In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent. For administration, cells of the present invention can be administered at a rate determined by the LD₅₀ of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces that may include, but are not restricted to, bone marrow or adipose tissues.

With regard to the therapeutic uses of the systems embodied herein, the off switch for deactivation of the endonuclease is important as it provides for a greatly enhanced targeting specificity of Cas9 while enabling for a controlled deactivation. Therefore, the present invention provides for a method of deactivating a gene editing agent comprising contacting a cell with a composition comprising a gene editing agent, wherein the gene-editing agent comprises a clustered regularly interspaced short palindromic repeats (CRISPR)-associated endonuclease, a Cas peptide and at least one guide RNA (gRNA) comprising at least one photocleavable linker molecule; subjecting the cell to an electromagnetic radiation, thereby cleaving the at least one gRNA and deactivating the gene-editing agent. In certain embodiments, the electromagnetic radiation comprises a wavelength of between about 190 to about 2400 nm. In certain embodiments, the electromagnetic radiation has a wavelength of about 365 nm. In certain embodiments, the at least one photocleavable linker molecule is located between 10 to 20 nucleotides distal to a protospacer adjacent motif (PAM). In certain embodiments, the photocleavable linker molecule is positioned at 15 nucleotides distal to the PAM, wherein cleavage truncates at a region of target complementarity to 14 nucleotide, rendering Cas9 cleavage-incompetent.

The therapeutic uses include, for example, virus infections, tumors, autoimmune diseases melanomas, and the like. The compositions can be utilized to edit a viral genome and inactivate the virus, e.g. HIV. The compositions can correct mutations, e.g. sickle cell anemia. In such cases, the compositions include a cleavable linker. The linker can be cleaved, thereby inactivating the endonuclease at a desired time in the therapy. A therapeutically effective amount of a composition (i.e., an effective dosage) can be delivered means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

The pharmaceutical compositions of the present invention can be prepared in a variety of ways known to one of ordinary skill in the art. Regardless of their original source or the manner in which they are obtained, the compositions of the invention can be formulated in accordance with their use. For example, the nucleic acids and vectors described above can be formulated within compositions for application to cells in tissue culture or for administration to a patient or subject. Any of the pharmaceutical compositions of the invention can be formulated for use in the preparation of a medicament, and particular uses are indicated below in the context of treatment, e.g., the treatment of a subject having an HIV infection or at risk for contracting and HIV infection. When employed as pharmaceuticals, any of the nucleic acids and vectors can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

Kits

The present invention also includes a kit to facilitate the application of the previously stated methods. The kit includes a measured amount of a composition including at least one isolated nucleic acid sequence encoding an endonuclease, and at least one nucleic acid sequence encoding one or more gRNAs, wherein each of the gRNAs includes at least one photocleavable linker. The kit also includes and one or more items selected from the group consisting of packaging material, a package insert comprising instructions for use, a sterile fluid, a syringe and a sterile container. In a preferred embodiment, the nucleic acid sequences are included in an expression vector. The kit can also include a suitable stabilizer, a carrier molecule, a flavoring, or the like, as appropriate for the intended use.

Accordingly, packaged products (e.g., sterile containers containing one or more of the compositions described herein and packaged for storage, shipment, or sale at concentrated or ready-to-use concentrations) and kits, including at least one composition of the invention, e.g., a nucleic acid sequence encoding an endonuclease, a guide RNA comprising at least one photocleavable linker, or a vector encoding that nucleic acid and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required.

The product may also include a legend (e.g., a printed label or insert or other medium describing the product's use (e.g., an audio- or videotape)). The legend can be associated with the container (e.g., affixed to the container) and can describe the manner in which the compositions therein should be administered and may include one or more additional pharmaceutically acceptable adjuvants, carriers or other diluents and/or an additional therapeutic agent. Alternatively, the compositions can be provided in a concentrated form with a diluent and instructions for dilution.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

### EXAMPLES

### Example 1: A CRISPR-Cas9 Platform with a Built-In Off-Switch and Enhanced Specificity

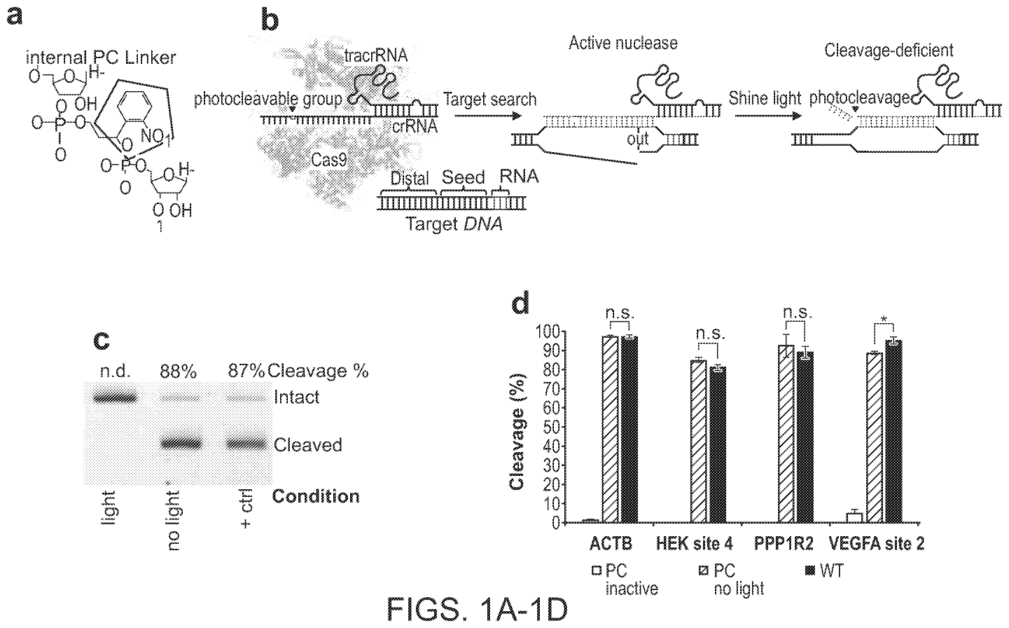

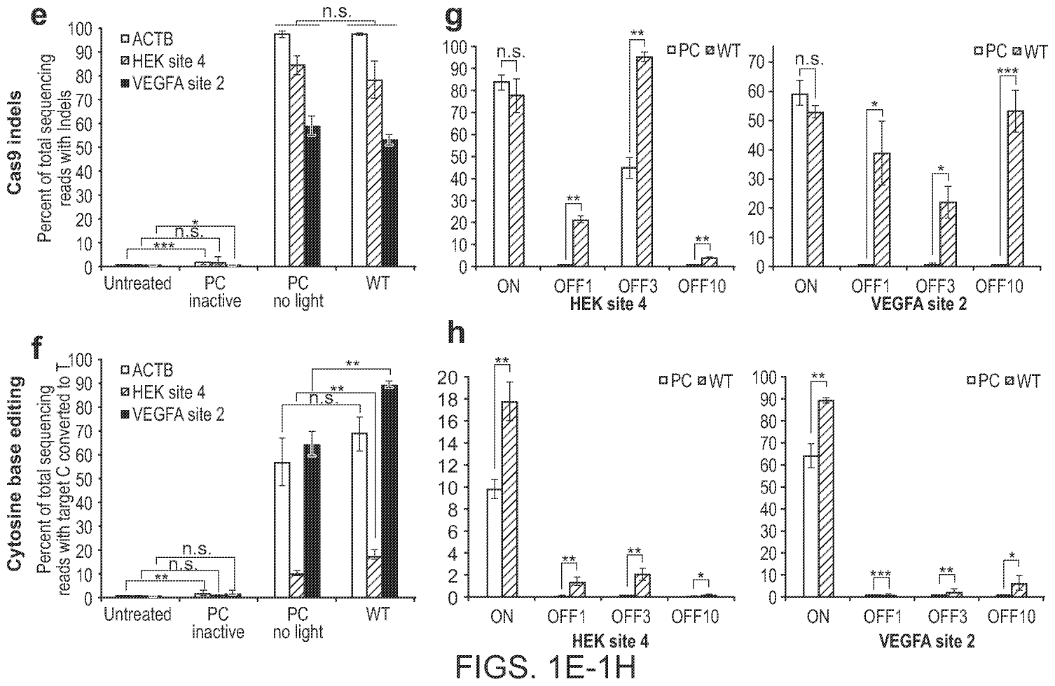

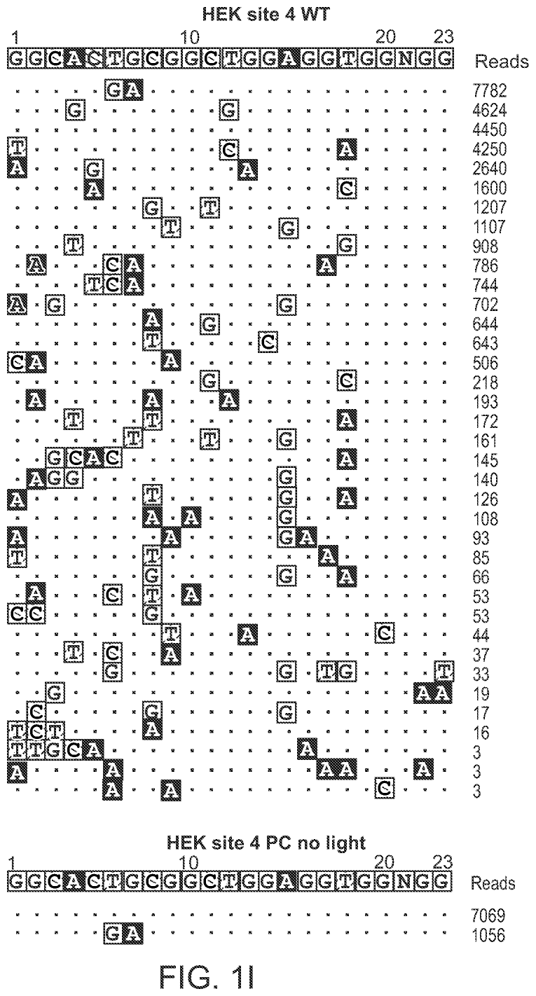

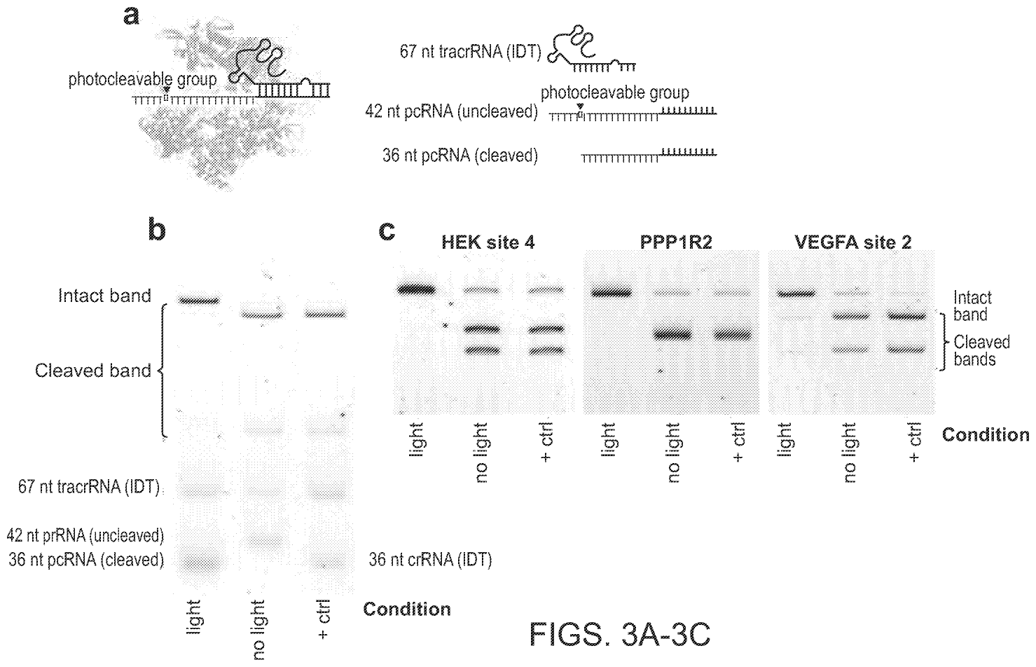

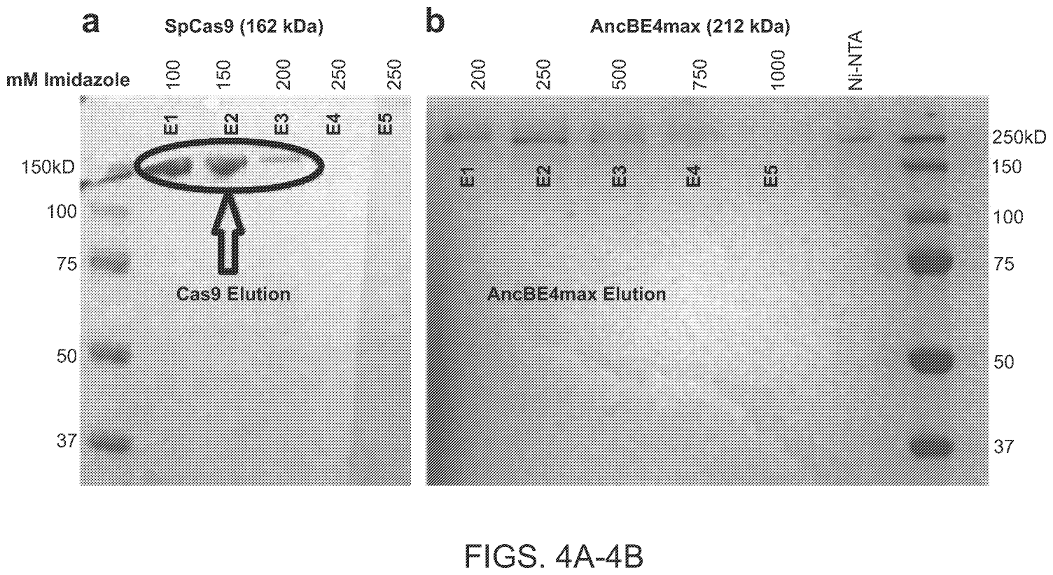

A CRISPR/Cas9 system with a built-in off-switch such that Cas9 can be rapidly and irreversibly deactivated with high spatiotemporal precision was engineered. To accomplish this, the 15th nucleotide (counting from PAM) of a full-length crRNA was replaced with a photocleavable 2-nitrobenzyl linker (PC-Linker), termed herein, a photocleavable guide RNA (pcRNA). It was hypothesized that pcRNA would minimally perturb the Cas9-gRNA complex and retain Cas9 cleavage competency, but brief illumination with a low dose of 365 nm wavelength light would cleave the chemical group, effectively truncating the region of target complementarity to 14 nucleotides and rendering Cas9 cleavage-incompetent (FIGS. 1A, 1B, 3A).

### Materials and Methods

Molecular Cloning:

Cloning pET42b-AncBE4max for protein expression and purification. The AncBE4max fragment from pCMV_AncBE4max_P2A_EGFP mammalian expression plasmid (Addgene #112100) was ligated to a pET42b vector backbone (Addgene #87438) using NEBUILDER® HiFi DNA Assembly Master Mix (New England BioLabs E2621) and transformed into NEB5α cells following manufacturer's instructions. Primer sequences Gib_pET42b_F and Gib_pET42b_R were used to PCR amplify the pET42b backbone, and primer sequences Gib_BEmax_F and Gib_BEmax_R were used to amplify AncBE4max.

Cloning pLPC-mCh-ACTB-P2A-EGFP for mCh/EGFP reporter of Cas9 cleavage activity in cells. The backbone with mCherry was obtained from restriction digest of mCherry-BP1-2 pLPC-Puro (Addgene 19835) with BamHI-HF (New England BioLabs). ACTB fragment was obtained from PCR of genomic DNA from HEK293T cells using primers ACTB_150nt_fwd and ACTB 150nt rev. P2A-EGFP fragment was obtained from PCR of pCMV_AncBE4max_P2A_EGFP (Addgene #112100) using primers P2A-EGFP_fwd and P2A-EGFP rev. The 3 pieces were ligated together with NEBuilder® HiFi DNA Assembly Master Mix (New England BioLabs E2621) and transformed into NEB5α cells following manufacturer's instructions. All primers sequences used for these two cloning projects are in Table 2.

### SpCas9 Purification

BL21-CodonPlus (DE3)-RIL competent cells (Agilent Technologies 230245) were transformed with Cas9 plasmid (Addgene #67881) and inoculated in 5 ml of LB-ampicillin media. The bacteria culture was first allowed to grow overnight (37° C., 220 rpm) and then transferred to 1 L of LB supplemented with ampicillin and 0.1% glucose until OD₆₀₀ of ˜0.5. Subsequently, the cells were induced with IPTG at a final concentration of 0.2 mM and maintained overnight at 18° C. The bacteria cells were pelleted at 4500×g, 4° C. for 15 min and resuspended in 20 ml of lysis buffer containing 20 mM Tris pH 8.0, 250 mM KCl, 20 mM imidazole, 10% glycerol, 1 mM TCEP, 1 mM PMSF, and cOmplete™ EDTA-free protease inhibitor tablet (Sigma-Aldrich 11836170001). This cell suspension was lysed using a microfluidizer and the supernatant containing Cas9 protein was clarified by spinning down cell debris at 16,000×g, 4° C. for 40 min and filtering with 0.2 μm syringe filters (Thermo Scientific™ F25006). Ni-NTA agarose bead slurry (Qiagen 30210) was pre-equilibrated with 5 column volumes of lysis buffer. The clarified supernatant was then loaded at 4° C. The protein-bound Ni-NTA beads were washed with 15 column volumes wash buffer containing 20 mM Tris pH 8.0, 800 mM KCl, 20 mM imidazole, 10% glycerol, and 1 mM TCEP. Gradient elution was performed with buffer containing 20 mM HEPES pH 8.0, 500 mM KCl, 10% glycerol, and varying concentrations of imidazole (100, 150, 200, and 250 mM) at 7 ml collection volume per fraction. The eluted fractions were tested on an SDS-PAGE gel and imaged by Coomassie blue (Bio-Rad 1610400) staining. To remove any DNA contamination, 1 ml Q SEPHAROSE® column (GE Healthcare 17051005) was charged with 1M KCl and then equilibrated with elution buffer containing 250 mM imidazole. The purified protein solution was then passed over the Q column at 4° C. The flow-through was collected and dialyzed in a 10 kDa SNAKESKIN™ dialysis tubing (Thermo Fisher Scientific 68100) against 2 L of 20 mM HEPES pH 7.5, and 500 mM KCl, 20% glycerol at 4° C., overnight. Next day, the protein was dialyzed for an additional 3 hours in fresh dialysis buffer. The final Cas9 protein was concentrated to 10 μg/μl using AMICON® Ultra 10 kDa centrifugal filter unit (Millipore UFC801024), aliquoted, and flash-frozen and stored at −80° C.

AncBE4max Purification

Protein expression and purification of AncBE4max was similar to that for SpCas9. BL21 Star (DE3) competent cells (Thermo Fisher Scientific C601003) were transformed and inoculated in 5 ml of LB-kanamycin media. The bacteria culture was first allowed to grow overnight (37° C., 220 rpm) and then transferred to 1 L of LB supplemented with kanamycin and 0.1% glucose until OD₆₀₀ of ˜0.7. Subsequently, the cells were induced with IPTG at a final concentration of 0.5 mM and maintained overnight at 18° C. The bacteria cells were pelleted at 4500×g, 4° C. for 15 min and resuspended in 20 ml of lysis buffer containing 100 mM Tris pH 8.0, 1M NaCl, 20% glycerol, 5 mM TCEP, 0.4 mM PMSF, and COMPLETE™ EDTA-free protease inhibitor tablet (Sigma-Aldrich 11836170001). This cell suspension was lysed by sonication (10%, 1.5s ON, 5s OFF, 10 min ON time) and the supernatant containing AncBE4max was clarified by spinning down cell debris at 16,000×g, 4° C. for 40 min and filtering with 0.2 μm syringe filters (THERMO SCIENTIFIC™ F25006). His Pur Ni-NTA agarose bead slurry (Thermo Fisher Scientific 88221) was pre-equilibrated with 5 column volumes of lysis buffer. The clarified supernatant was then loaded at 4° C. The protein-bound Ni-NTA beads were washed with 15 column volumes wash buffer containing 100 mM Tris-HCl pH 8.01M NaCl, 20% glycerol, and 5 mM TCEP. Gradient elution was performed with buffer containing 100 mM Tris-HCl pH 8.0, 500 mM NaCl, 20% glycerol, 5 mM TCEP, and varying concentrations of imidazole (200, 250, 500, 750, 100 mM) at 7 ml collection volume per fraction. The eluted fractions were tested on an SDS-PAGE gel and imaged by Coomassie blue (Bio-Rad 1610400) staining. To remove any DNA contamination, 1 ml Q SEPHAROSE® column (GE Healthcare 17051005) was charged with 1M KCl and then equilibrated with elution buffer containing 250 mM imidazole. The purified protein solution was then passed over the Q column at 4° C. The flow-through was collected and dialyzed in a 10 kDa SNAKESKIN′ dialysis tubing (Thermo Fisher Scientific 68100) against 2 L of 25 mM HEPES pH 7.5, and 500 mM KCl, 20% glycerol at 4° C., overnight. Next day, the protein was dialyzed for an additional 3 hours in fresh dialysis buffer. The final protein was concentrated to 10 μg/μl using AMICON® Ultra 10 kDa centrifugal filter unit (Millipore UFC801024), aliquoted, and flash-frozen and stored at −80° C.

Cell Culture

Human embryonic kidney 293 cell line (HEK293T) were cultured at 37° C. under 5% CO₂ in Dulbecco's Modified Eagle's Medium (DMEM, Corning), supplemented with 10% FBS (Clontech), 100units/ml penicillin and 100 μg/ml streptomycin (DMEM complete). Cells were tested every month for mycoplasma.

Electroporation of SpCas9/AncBE4max RNP

To anneal crRNA (either photocleavable or wild type) with tracrRNA, equal volumes of 100 μM crRNA with tracrRNA (Integrated DNA Technologies) were mixed and heated to 95° C. for 5 min in a thermocycler. The mixture was allowed to cool on benchtop for 5 min. To form RNP complex, 10 μg/μl of purified Cas9 or AncBE4max was mixed with 50 μM cr:tracrRNA at a ratio of 1:1.2, which was then incubated for additional 20 min at room temperature. Cells were properly maintained to a confluency of ˜90% prior to electroporation. Cells were then trypsinized and centrifuged in DMEM and 1×PBS sequentially (3 min, 200 g). Supernatant was discarded and 20 μL of nucleofection solution (Lonza) was mixed thoroughly with cell pellet, prior to the addition of 5 μL RNP solution. 1 μL of Cas9 Electroporation Enhancer (Integrated DNA Technologies) was also included. Electroporation was performed according to the manufacturer's instructions on the 4D-Nucleofector™ Core Unit (Lonza). SF Cell Line 4D-Nucleofector™ X Kit S with code CA-189 was used for HEK293T cells. DMEM complete was added before plating to culture wells.

Preparing Samples for Kinetics Measurements in Cells

HEK 293T cells were introduced with SpCas9/AncBE4max in complex with pcRNA through electroporation, plated to 96-wells, and incubated in standard cell culture conditions. At various time points, cells were exposed to a flashlight that delivered 1.3 J/cm2 of 365 nm wavelength light. For pre-cleaved pcRNA, light was delivered to the RNP complex before electroporation. 72 h after electroporation, cells were harvested with DPBS and genomic DNA was isolated using DNeasy Blood & Tissue Kit (Qiagen 69506) according to the manufacturer's instructions, except with 1 h (instead of 10 min) incubation with lysis buffer/Proteinase K at 55° C.

Sanger Sequencing for Measuring Insertions or Deletions

Genomic DNA samples were amplified with PCR using Q5 Hot Start High-Fidelity 2× Master Mix (New England BioLabs M0494). The primer pairs and PCR amplification conditions are listed in Tables 3, 6, 7. After PCR, cleanup was performed using 1.5×AMPure XP (Beckman Coulter A63881) following the manufacturer's instructions. 3 ng/μl of each sample was submitted to Genewiz for Sanger sequencing. Indels were calculated using TIDE analysis (Brinkman E K, Chen T, Amendola M, van Steensel B. Easy quantitative assessment of genome editing by sequence trace decomposition. Nucleic Acids Res. 2014 Dec. 16; 42(22):e168. doi: 10.1093/nar/gku936. Epub 2014 Oct. 9. PMID: 25300484; PMCID: PMC4267669).

High Throughput Sequencing of Genomic DNA Samples

Genomic DNA samples were amplified with PCR using Q5 Hot Start High-Fidelity 2× Master Mix (New England BioLabs M0494). All primer pairs and PCR amplification conditions are listed in Tables 4-7. After amplicon PCR, cleanup was performed using 1.6× AMPure XP (Beckman Coulter A63881) following the manufacturer's instructions. Dual-indexing PCR was performed using KAPA HiFi HotStart ReadyMix (Roche 07958935001) and PCR cleanup was performed using 1×AMPure XP. Samples were quantified using QuBit (Thermo Fisher Scientific), pooled, diluted, and loaded onto a MiSeq (Illumina). Sequencing was performed with the following number of cycles “151|8|8|151” with the paired-end Nextera sequencing protocol.

Data Analysis of HTS Results

Sequencing reads were either demultiplexed automatically using MiSeq Reporter (Illumina) or with a custom Python script to individual FASTQ files. This script also performs indel and base calling. For indel calling, sequencing reads were scanned for exact matches to two 20-bp sequences that flank+/−20 bp from the ends of the target sequence. If no exact matches were found, the read was excluded from analysis. After additional filtering for an average quality score>20, an indel is defined as a sequence that differs in length from the reference length. For base calling, sequencing reads were scanned for exact matches to two 20 bp sequences that flank the target sequence. If no exact matches were found, or the match led to sequences of different length compared to the reference sequence, the read was excluded from analysis. Any base with quality score>30 was counted.

In Vitro Cleavage Assay

Target DNA are amplified from genomic DNA using primers designed for Sanger sequencing (Tables 3, 6, 7), and purified with QIAQuick PCR Purification Kit (Qiagen). 10 cr:tracrRNA solution was prepared at equal molar ratio by heating to 95° C. for 5 min and cooling on a heat block for 1 hour. Either photocleavable crRNA or wild type crRNA were used to mix with tracrRNA to form Cas9-pcRNA or Cas9-wtRNA, respectively (all purchased from Integrated DNA Technologies). 3 pmol of Cas9 was incubated with 5 pmol of gRNA to form RNP for 30 min in 10 μl of 1×NEBuffer 3.1 (New England Biolabs). The tube was placed on a 37° C. heat block for 1 min, 365 nm light was applied for 30 sec, then 60 fmol of target DNA was added and thoroughly mixed. A no light control omits application of light, and a positive control uses the wild type crRNA. To demonstrate light-induced deactivation, all samples were incubated for 1 h at 37° C. To evaluate the kinetics of cleavage, Cas9-pcRNA samples were incubated for 30 sec, 1 min, 2 min, 5 min, 10 min, 30 min, 45 min, 1 h from the time DNA was added and Cas9-wtRNA samples were incubated for 30 sec, 1 min, 2 min, 5 min, and 1 h from the time DNA was added. After incubation, 10 μg of Proteinase K (Thermo Fisher) was added to each tube and further incubated in 55° C. for 45 min. The DNA was then purified with QIAquick PCR Purification Kit (Qiagen) before loading on an agarose gel for visualization. To calculate the cleavage efficiency, the integrated intensity of cleaved bands was divided by that of total DNA as quantified using ImageJ.

GUIDE-seq

HEK293T cells were electroporated with the protocol described previously, with an additional 25 pmol dsODN mixed with the RNP. 72 h after electroporation, cells were harvested with DPBS and genomic DNA was isolated using DNeasy Blood & Tissue Kit (Qiagen 69506) according to the manufacturer's instructions, except with 1 h (instead of 10 min) incubation with lysis buffer/Proteinase K at 55° C. Library preparation was done with the corrected adaptor sequences described in Tsai, S. Q., et al. GUIDE-seq enables genome-wide profiling of off-target cleavage by CRISPR-Cas nucleases. Nature Biotechnology 33, 187-197 (2015)) The library was quantified with qPCR using NEBNEXT® Library Quant Kit for Illumina® (New England BioLabs E7630), pooled, diluted, and loaded onto a MiSeq (Illumina). Sequencing was performed with the following number of cycles “150|8|16|150” with the paired-end Nextera sequencing protocol following the protocol described in Tsai, S. Q., et al. 2015. Data analysis was done using code adapted from github.com/aryeelab/guideseq, with the original filter for sequences containing six or fewer mismatches between candidate off-target sites and the on-target sequence including the NGG PAM.

Spatial Control of Cas9 Deactivation

HEK293T cells were electroporated with SpCas9-pcRNA targeted ACTB using the electroporation protocol described earlier, and plated on 14 mm glass-bottom dishes (Cellvis D35-14-1.5-N). 12 h later, cells were illuminated using a LED light from the bottom of the dish with a mask between the cells and LED. Cells were transfected with GFP reporter plasmid within 30 min of light delivery. Fluorescence imaging for GFP was performed 24 h later using 10× air objective on a Nikon Ti-E fluorescence microscope equipped with an Andor EMCCD.

Immunofluorescence Microscopy

HEK293T cells were electroporated with SpCas9-wtRNA targeting ACTB using the electroporation protocol described earlier, and plated on 20 mm glass-bottom dishes (Cellvis D35-20-1.5-N). 1 h later after cells are adherent to surface, cell fixation was performed with 4% of paraformaldehyde in 1×PBS for 10 min and then quenched by 1×PBS supplemented with 0.1 M glycine for 10 min. After thoroughly rinsing with 1×PBS, 0.5 Triton-X was used to permeabilize cell membrane for 10 min. 2% w/v BSA in 1×PBS was used to passivate the sample for 1 h and at room temperature. Without further rinsing, primary antibody was diluted in 1×PBS and directly added into the chamber for targeting the protein of interests. After 1 h incubation, primary antibody was removed and the sample was thoroughly washed with 1×PBS three times. Secondary antibody was typically diluted in 1:1000 and applied to the sample for 1 h. Finally, the sample was rinsed three times and mounted with Prolong Diamond mounting media (Thermo Fisher Scientific) overnight. Electroporation efficiency was estimated from manual counting of Cas9 positive-cells using a hemocytometer. Mouse anti-SpCas9 (7A9-3A3) was purchased from Cell Signaling Technology (14697). Cy5 conjugated Goat anti-mouse antibody (A10524) was purchased from Thermo Fisher Scientific. Dilution of primary antibody was based on the recommended ratio from the manufacturers.

Statistics

Student's t-tests were used to calculate P values on Excel (Microsoft). P<0.05 is considered significant. The definition of error bars is standard deviations. N indicates biological replicates. No inclusion and exclusion criteria of samples were used.

### Results

To validate that Cas9 deactivation occurs within seconds using in vitro cleavage of four different synthetic DNA sequences, the following experiments were conducted. Purified SpCas9 (FIG. 4A) in complex with pcRNA was illuminated with a LED light source (Jaxman, 365 nm, ˜40 mW/cm², 30 s), and target DNA was added within 45 s from the start of light illumination. After incubation at 37° C. for 1 h, there was almost no detectable cleavage of target DNA for samples exposed to light but high cleavage efficiency for samples without light, comparable to that of Cas9 with wild type gRNA (wtRNA) (FIGS. 1C, 1D, 3B, 3C). The activity of pcRNA in HEK293T cells was next validated using three target sequences (ACTB, HEK site 4 and VEGFA site 2) (Tsai, S. Q., et al. GUIDE-seq enables genome-wide profiling of off-target cleavage by CRISPR-Cas nucleases. Nature Biotechnology 33, 187-197 (2015)). Percent of insertions and deletions (indels) 3 days after Cas9-pcRNA delivery by electroporation was computed from a combination of Sanger Sequencing/TIDE analysis (Brinkman, E. K., et al. Easy quantitative assessment of genome editing by sequence trace decomposition. Nucleic Acids Research 42, 168 (2014) and targeted deep sequencing. It was found that application of light immediately before delivery reduced indels to almost undetectable levels. In contrast, cells without light exposure had indel efficiencies comparable to those obtained with wtRNA (FIG. 1E).

The pcRNA should also be compatible with other platforms that utilize the Cas9 component such as single-nucleotide base editors, which only function effectively upon full DNA unwinding and target-strand nicking; truncated guides retain binding but inhibit base editing presumably due to the lack of full unwinding and nickase activity (Komor, A. C., et al. Programmable editing of a target base in genomic DNA without double-stranded DNA cleavage. Nature 533, 420-424 (2016); Kim, Y. B., et al. Increasing the genome-targeting scope and precision of base editing with engineered Cas9-cytidine deaminase fusions. Nature Biotechnology 35, 371-376 (2017)). Thus, it was investigated whether the system herein enables light-mediated deactivation of Cas9-dependent DNA editing by base editors. In a scheme similar to that for Cas9, purified AncBE4max (FIG. 4B) (Koblan, L. W., et al. Improving cytidine and adenine base editors by expression optimization and ancestral reconstruction. Nature Biotechnology 36, 843-846 (2018)) in complex with pcRNA was either illuminated with light right before electroporation or not given light. The percent of base editing was calculated from targeted deep sequencing 3 days later. At the same three endogenous loci, almost complete suppression of base editing with light and high efficiency base editing without light was observed (FIG. 1F).

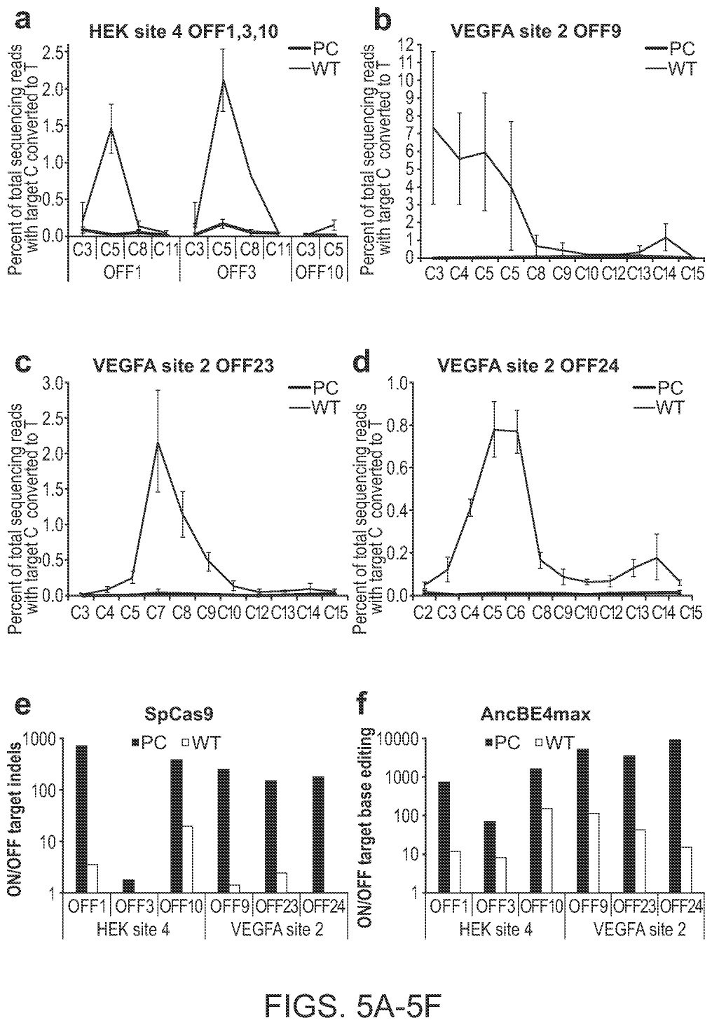

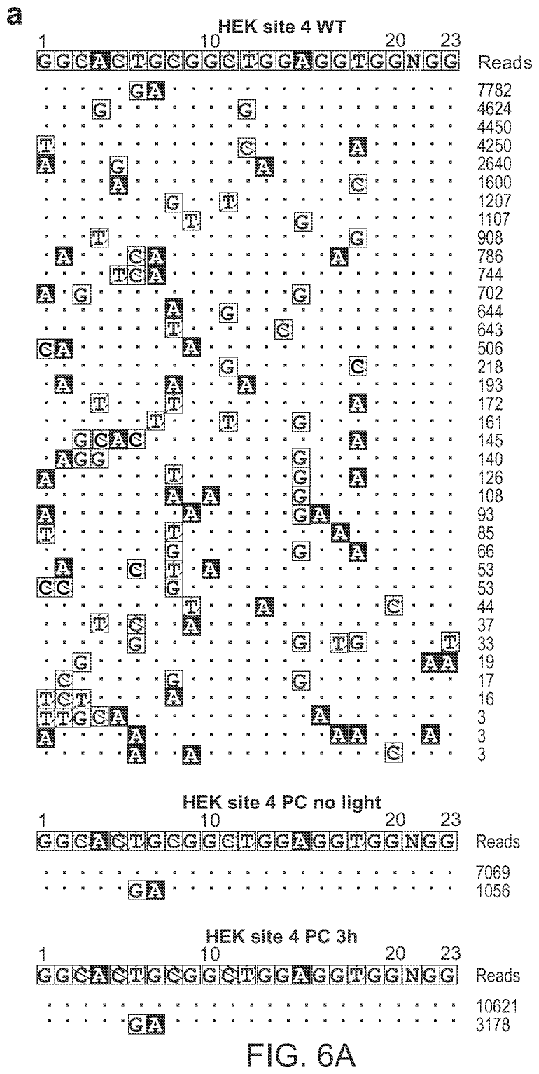

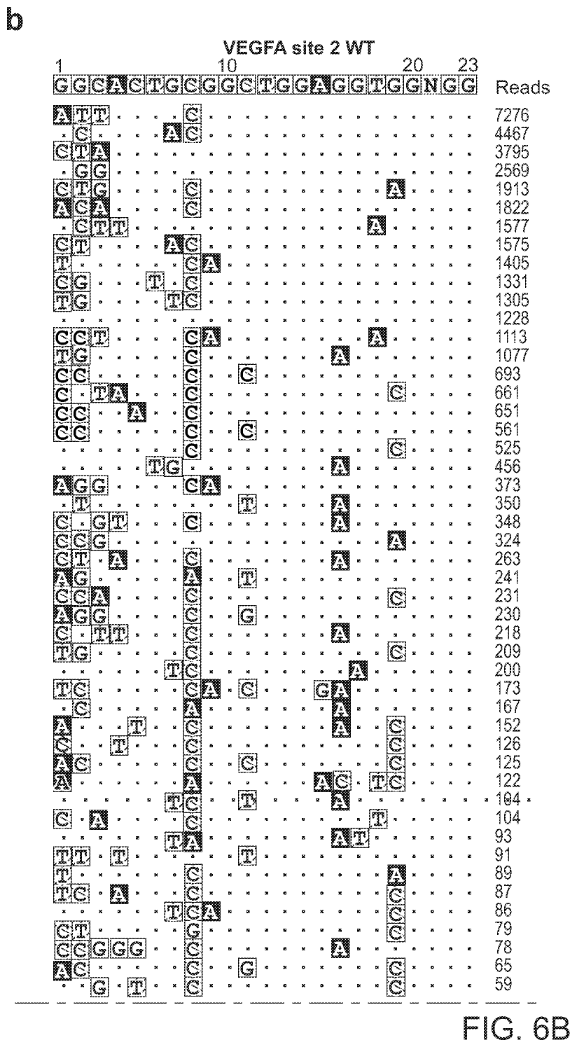

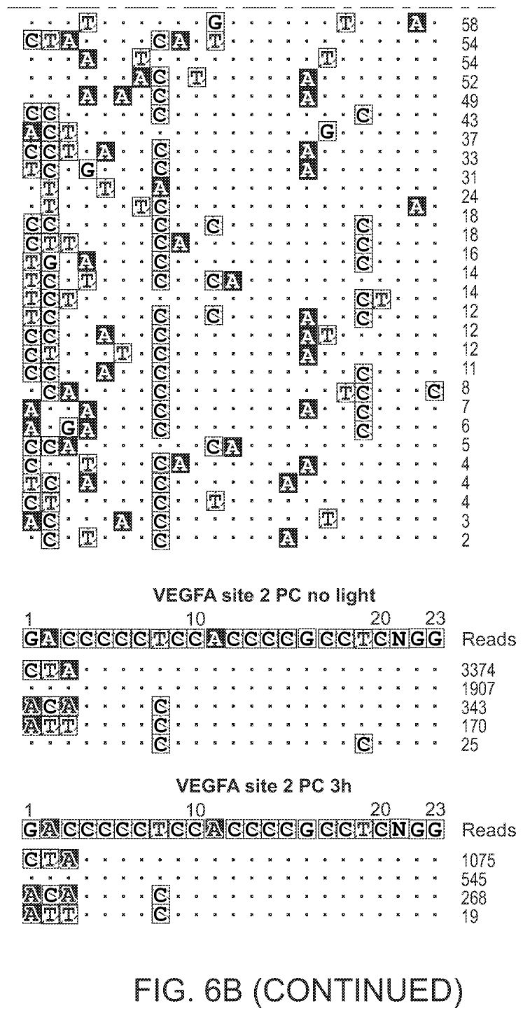

Two target sequences that were tested, VEGFA site 2 and HEK site 4, have many well-validated off-target sites. Indels and cytosine base editing was measured at select off-target sites to test whether the chemical group in our gRNA natively, that is, without photocleavage, modulates targeting specificity. Interestingly, dramatic suppression of Cas9-mediated off-target indels and AncBE4max-mediated off-target base editing was observed (FIGS. 1G, 1H, 5A-5D). Compared to wtRNA, the use of pcRNA improved the ratio of on-target to off-target editing by 10 to 1000-fold at the off-target sites tested (FIGS. 5E, 5F). To determine if the improved specificity holds genome-wide, we performed GUIDE-seq (Tsai, S. Q., et al. GUIDE-seq enables genome-wide profiling of off-target cleavage by CRISPR-Cas nucleases. Nature Biotechnology 33, 187-197 (2015)) on these two target sequences. A large reduction was observed in off-target sites with pcRNA compared to wtRNA, with 1 versus 36 off-target sites for HEK site 4 (97% reduction), and 4 versus 76 off-target sites for VEGFA site 2 (95% reduction), respectively (FIGS. 1I, 6A, 6B). This is the biggest reduction in off-target sites that has been reported in literature for both target sequences (Chen, J. S., et al. Enhanced proofreading governs CRISPR-Cas9 targeting accuracy. Nature 550, 407-410 (2017). Collectively, the data provide evidence that pcRNA itself endows wild type SpCas9 with significantly enhanced specificity and uncompromised on-target activity in cells.

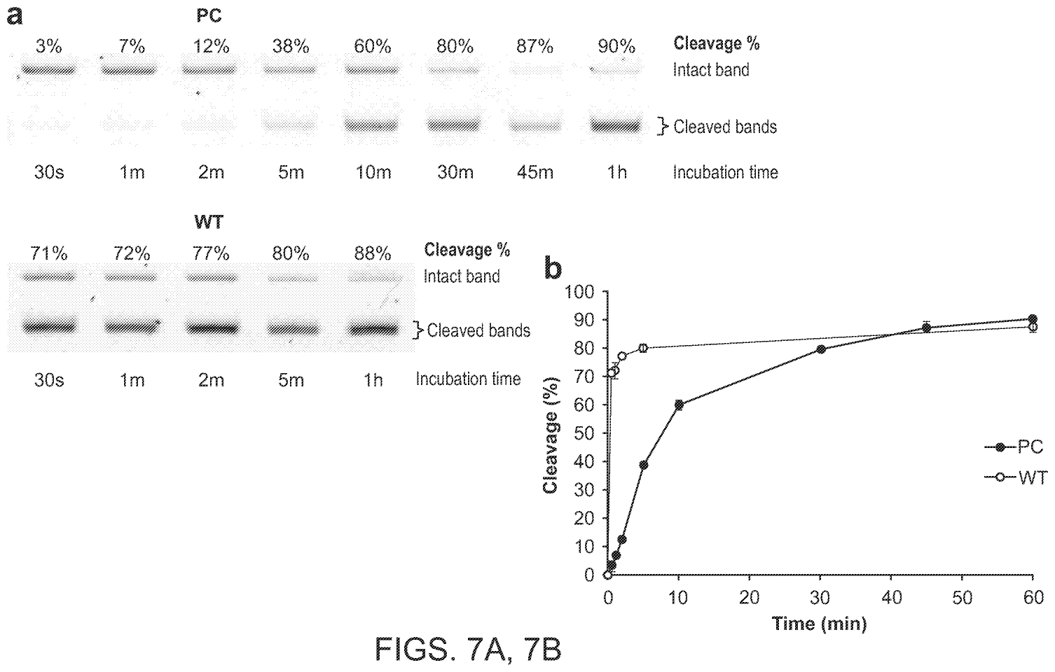

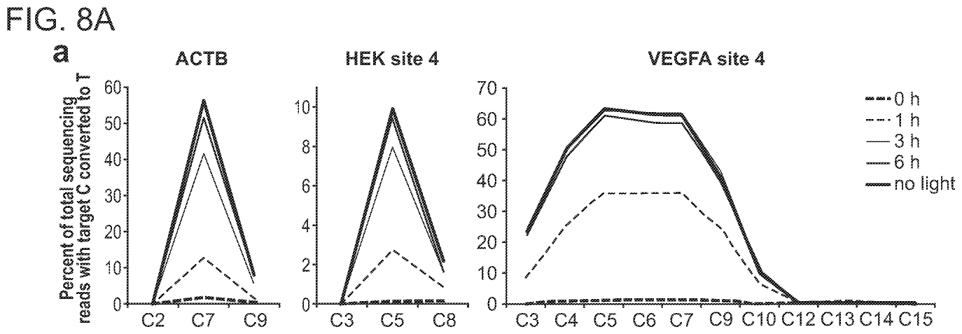

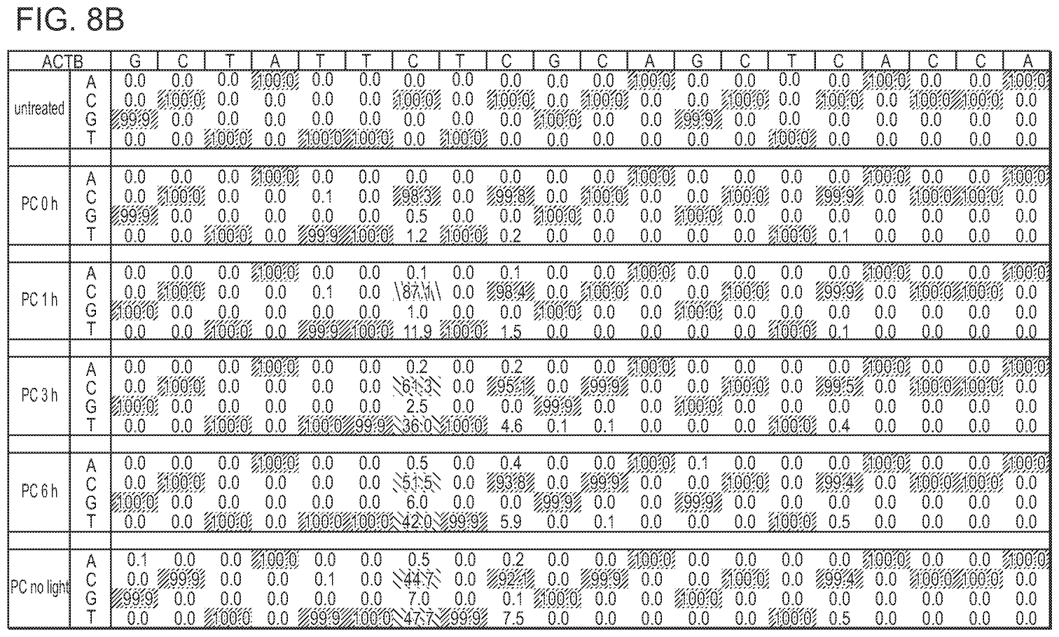

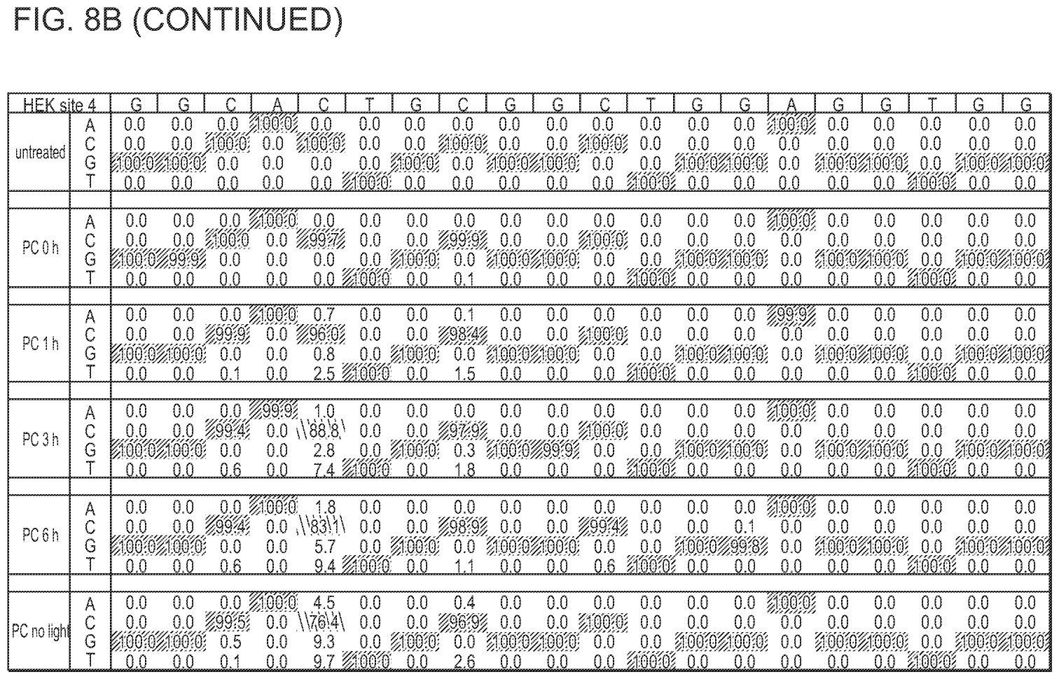

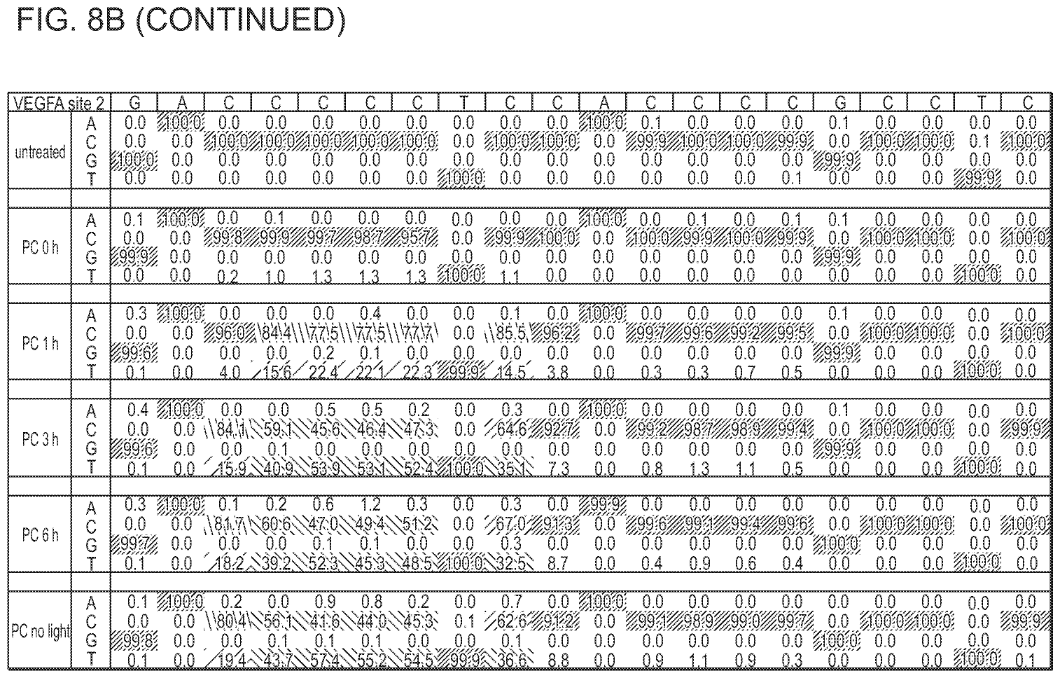

To gain insight on why pcRNA itself improves specificity, in vitro cleavage kinetics were measured. Cas9-pcRNA required over 10 min to achieve comparable cleavage efficiencies as a 30 sec reaction with Cas9-wtRNA, even though the final cleavage efficiencies were similar (FIGS. 7A, 7B). Without wishing to be bound by theory, it was speculated that the photocleavable group may interfere with the directional R-loop formation process such that achieving cleavage competency takes longer and are more sensitive to mismatches, a mechanism similar to that proposed for other design strategies that enhance specificity (e.g. modified gRNA, rationally designed enzymes) (Vakulskas, C. A., et al. Nature Medicine 24, 1216-1224 (2018); Kleinstiver, B. P., et al. Nature 529, 490-495 (2016); Slaymaker, I. M., et al. Science 351, 84-88 (2016); Kocak, D. D., et al. Nature Biotechnology 37, 657-666 (2019); Yin, H., et al. Nature Chemical Biology 14, 311-316 (2018); Cromwell, C. R., et al. Nature Communications 9, 1448 (2018)).

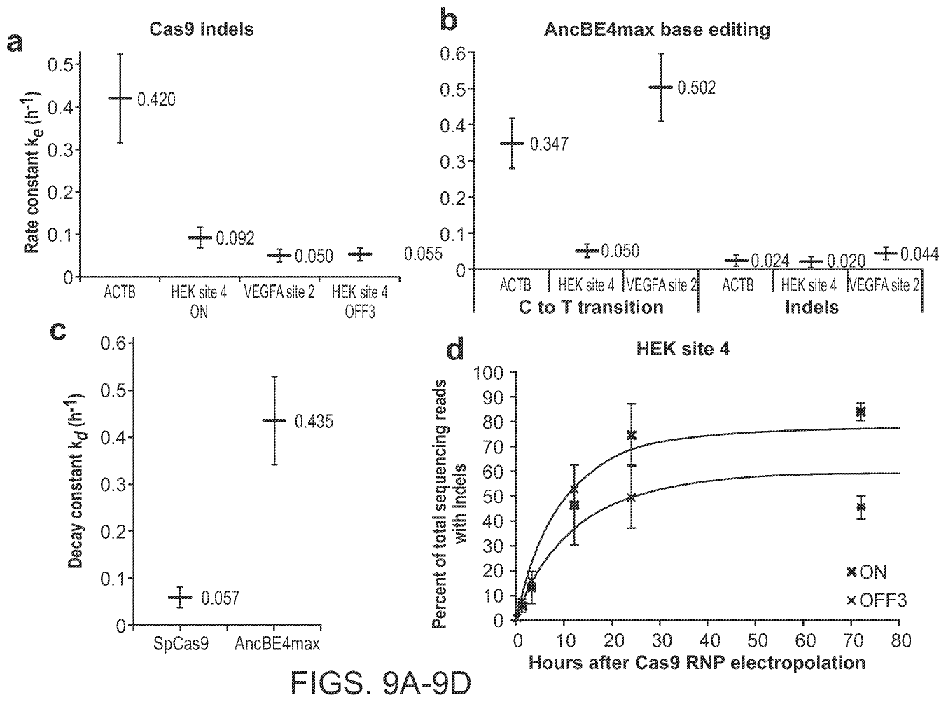

The effect on final genome editing was investigated at 72 h from deactivating either Cas9 or base editor at various time points after delivery into cells (FIG. 2A). Shining light at early time points greatly reduced the final genome editing whereas allowing more time before deactivation led to increased editing (FIGS. 2B, 2C). Furthermore, differences were observed in duration-dependent editing efficiencies between indel formation and base editing. For example, VEGFA site 2 showed the highest base editing efficiency but lowest indel efficiency among the three loci. To quantify these differences, a mathematical model was developed whereby varying the duration of CRISPR activity modulates “conversion” of the final sequence from unmodified to edited with rate constant Kₑ (see, Example 2). After fitting all data to this model (FIGS. 2B-2D, 9A-9D), it was found that Kₑ varied greatly between loci and between SpCas9 and AncBE4max. For Cas9 indels, Kₑ was the highest for ACTB and lowest for VEGFA site 2. In contrast, for AncBE4max base editing, Kₑ was the highest for VEGFA site 2 and lowest for HEK site 4. Interestingly, the rank order of Kₑ values for AncBE4max-induced indels (FIGS. 2D, 9B) matched that for base editing but not Cas9-induced indels. Collectively, the results highlight clear locus dependence in base editing that is distinct from Cas9 nucleases. Assuming that target searching rates and local sequence accessibility for each locus is comparable between SpCas9 and AncBE4max, this implies that DNA repair processes are primarily responsible for the observed duration-dependent differences.

Limiting the duration of CRISPR activity, as demonstrated in literature using anti-CRISPR proteins, has the potential advantage of limiting the chance of off-target genome editing (Shin, J., et al. Disabling Cas9 by an anti-CRISPR DNA mimic. Science Advances 3, 7 (2017)). To test whether the light-induced deactivation system can achieve a similar effect, GUIDE-seq was performed on cells with Cas9-pcRNA deactivated with light after 3 h of activity and genomic DNA harvested after 72 h. Compared to samples without light, it was found that limiting Cas9 to 3 h of activity did not reduce the number of off-target sites for HEK site 4, and reduced just one off-target site for VEGFA site 2 (FIGS. 6A, 6B). However, even for the one HEK site 4 off-target site (OFF3) that still had appreciable indel activity with pcRNA, limiting the activity window of Cas9 did not improve specificity (FIG. 9D). Therefore, the specificity benefit from stopping Cas9 early may not be a general phenomenon.

Next, the effect of temporally limiting base editor activity to the first 3 h on the final base editing at 72 h was examined. While investigating the kinetics of off-target base-editing was challenging due the enhanced baseline specificity of the pcRNA system, light-induced deactivation increased final base editing purity at ACTB and HEK site 4 (VEGFA site 2 natively had >99% purity) (FIG. 2E). Purity is defined as the fraction of final thymine, divided by the fraction of thymine, adenine, and guanine, at the targeted nucleotide (Rees, H. A., and Liu, D. R. Base editing: precision chemistry on the genome and transcriptome of living cells. Nature Reviews Genetics 19, 770-788 (2018)). The uracil-glycosylase inhibitor (UGI) domains of base editors are designed to significantly improve product purity by inhibiting base excision repair, but the modeling herein provides evidence of rapid cellular removal of active base editor (FIG. 9C), leading to reduced global UGI concentrations before the desired DNA repair processes (mismatch repair) is completed. This may lead to more repair through the base excision pathway and decreased product purity (Rees, H. A., and Liu, D. R. Base editing: precision chemistry on the genome and transcriptome of living cells. Nature Reviews Genetics 19, 770-788 (2018)) whereas stopping base editor early prevents new deamination events and may allow completion of the desired repair processes before the UGI-containing base editor is depleted through degradation.

Finally, the method was tested to determine whether it can achieve spatially defined Cas9 deactivation. Cas9-pcRNA was delivered to HEK293T cells, waited 12 h before shining light in a complex pattern, then transfected cells with an EGFP reporter plasmid containing the Cas9 cleavage site (FIG. 2F). After 24 h incubation, only cells exposed to light exhibited high EGFP expression, consistent with the expectation that after Cas9 deactivation, the reporter plasmid cannot be cleaved (FIG. 2G).

The pcRNA system combines high editing efficiency and superior targeting specificity with rapid and effective spatiotemporal control of both Cas9 nuclease and base editor deactivation. These improvements across multiple dimensions of genome editing are enabled by the rational replacement of just a single nucleotide. Many applications of this system are envisioned. Further insights into its mechanism for specificity improvement may guide engineering of the next generation enhanced-specificity CRISPR platforms. Rapid and spatially confined deactivation of a functional enzyme with light may enable new levels of control in synthetic biological circuits. Greatly reduced off-target editing with a fast, built-in off-switch is a synergistic safety mechanism for potential use in animal models and therapeutic applications.

TABLE 1
crRNA and tracrRNA sequences

| Name | Sequence |
|---|---|
| tracrRNA | AGCAUAGCAAGUUAAAAUAAGGCUAG |
| | UCCGUUAUCAACUUGAAAAAGUGGCA |
| | CCGAGUCGGUGCUUU (SEQ ID NO: 1) |
| Pcl5_ | GCU AU/iPC-Linker/CUC GCA GCU |
| ACTB | CAC CAG UUU UAG AGC |
| | UAU GCU GUU UUG (SEQ ID NO: 2) |
| Pcl5_ | GGC AC/iPC-Linker/GCG GCU GGA |
| HEKsite4 | GGU GGG UUU UAG AGC UAU GCU |
| | GUU UUG (SEQ ID NO: 3) |
| Pcl5_ | GAC UU/iPC-Linker/CUC UAU |
| PPP1R2 | GGU GGC GUG UUU UAG AGC |
| | UAU GCU GUU UUG (SEQ ID NO: 4) |
| Pcl5_ | GAC CC/iPC-Uinker/CUC CAC CCC |
| VEGFAsite2 | GCC UCG UUU UAG AGC UAU |
| | GCU GUU UUG (SEQ ID NO: 5) |
| IDT ACTB | GCU AUU CUC GCA GCU CAC CAG |
| | UUU UAG AGC UAU GCU |
| | (SEQ ID NO: 6) |
| IDT_ | GGC ACU GCG GCU GGA GGU GGG |
| HEKsite4 | UUU UAG AGC UAU GCU |
| | (SEQ ID NO: 7) |
| IDT_ | GAC UUC CUC UAU GGU GGC GUG |
| PPPIR2 | UUU UAG AGC UAU GCU |
| | (SEQ ID NO: 8) |
| IDT_ | GAC CCC CUC CAC CCC GCC UCG |
| VEGFAsite2 | UUU UAG AGC UAU GCU |
| | (SEQ ID NO: 9) |

tracrRNA was purchased as Alt-R® CRISPR-Cas9 tracrRNA from Integrated DNA Technologies (IDT);
Pcl5_* crRNAs were purchased as 42 nt (including PC Linker group) non-catalog orders from IDT;
IDT* crRNAs were purchased as 36 nt Alt-R® CRISPR-Cas9 crRNAs from IDT;
The bolded bases hybridize with target DNA.

TABLE 2
DNA sequences for cloning

| Name | Sequence (5′ to 3′) |
|---|---|
| Gib_pET42b_F | CCCAAGAAGAAGAGGAAAGTCT |
| | AATAATTG (SEQ ID NO: 10) |
| Gib_pET42b_R | GTGATGGTGATGATGATGACTG |
| | (SEQ ID NO: 11) |
| Gib_BEmaxF | gggcagcagtcatcatcatcacc |
| | atcacCCAAAGAAGAAGCGGAAA |
| | GTC (SEQ ID NO: 12) |
| Gib_BEmaxR | attagactttcctcttcttcttgg |
| | gCTCGAATTCGCTGCCGTC |
| | (SEQ ID NO: 13) |
| ACTB_150nt_ | catggacgagctgtacaagggatc |
| fwd | cGGCGGCCTAAGGACTCGG |
| | (SEQ ID NO: 14) |
| ACTB_150nt_ | ctgaagttagtagctccgctGAAG |
| rev | CCGGCCTTGCACATG |
| | (SEQ ID NO: 15) |
| P2A-EGFP_ | AGCGGAGCTACTAACTTC |
| fwd | (SEQ ID NO: 16) |
| P2A-EGFP_ | ggtcggcgcgcccacccttggatc |
| rev | ctcaCTTGTACAGCTCGTCCATG |
| | (SEQ ID NO: 17) |

TABLE 3
Amplicon PCR primers for Sanger sequencing
and generating target DNA for
in vitro cleavage assays

| Name | Sequence (5′ to 3′) |
|---|---|
| ACTB_F | TGGCGGCCTAAGGACTCG (SEQ ID NO: 18) |
| ACTB_R | CTTCAGGGTGAGGATGCCTCTC (SEQ ID NO: 19) |
| HEKs4_F | CCAGTGGTTCAATGGTCATCC (SEQ ID NO: 20) |
| HEKs4_R | GGCCAGTGAAATCACCCTG (SEQ ID NO: 21) |
| PPP1R2_F | GTTTCCGAGGCAGCAGTTG (SEQ ID NO: 22) |
| PPP1R2_R | GCATGATAAACGTCATCGCCC (SEQ ID NO: 23) |
| VEGFAs2_F | AGAGAAGTCGAGGAAGAGAGAG (SEQ ID NO: 24) |
| VEGFAs2_R | CAGCAGAAAGTTCATGGTTTCG (SEQ ID NO: 25) |

TABLE 4
Amplicon PCR primers for next
generation sequencing

| Name | Sequence (5′ to 3′) |
|---|---|
| NGSACTBF | tcgtcggcagcgtcagatgtgtataagaga |
| | cagTGGCGGCCTAAGGACTCG (SEQ ID |
| | NO: 26) |
| NGSACTBR | gtctcgtgggctcggagatgtgtataagag |
| | acagGAAGCCGGCCTTGCACATG (SEQ |
| | ID NO: 27) |
| NGS_HEKs4_ | tcgtcggcagcgtcagatgtgtataagaga |
| ON_F | cagGGTCCAAAGCAGGATGACAG (SEQ |
| | ID NO: 28) |
| NGS_HEKs4_ | gtctcgtgggctcggagatgtgtataaga |
| ON_R | gacagGAGACACACACACAGGCCT (SEQ |
| | ID NO: 29) |
| NGS_HEKs4_ | tcgtcggcagcgtcagatgtgtataagag |
| OFF1_F | acagCACTGCTCTCCAGAGTGGT (SEQ |
| | ID NO: 30) |
| NGS_HEKs4_ | gtctcgtgggctcggagatgtgtataaga |
| OFF1_R | gacagGATTCTCCTACTTCCTCCTCGG |
| | (SEQ ID NO: 31) |
| NGS_HEKs4_ | tcgtcggcagcgtcagatgtgtataagag |
| OFF3_F | acagGAGAAAAGCCAACGGGTTCTC |
| | (SEQ ID NO: 32) |
| NGS_HEKs4_ | gtctcgtgggctcggagatgtgtataaga |
| OFF3_R | gacagCATTGTCCCAGCTAAGCTCTCA |
| | (SEQ ID NO: 33) |
| NGS_HEKs4_OFF_ | tcgtcggcagcgtcagatgtgtataagag |
| 10F | acagCCCTGAGAAGGTAGTAGGAATCC |
| | (SEQ ID NO: 34) |
| NGS_HEKs4_OFF_ | gtctcgtgggctcggagatgtgtataaga |
| 10R | gacagGTGGTTAAGAGCAGACTCCCT |
| | (SEQ ID NO: 35) |
| NGS_VEGFAs2_ | tcgtcggcagcgtcagatgtgtataagag |
| ON_F | acagCTCCTCCGAAGCGAGAA (SEQ ID |
| | NO: 36) |
| NGS_VEGFAs2_ | gtctcgtgggctcggagatgtgtataaga |
| ON_R | gacagGACAGACAGACAGACACCG (SEQ |
| | ID NO: 37) |
| NGS_VEGFAs2_ | tcgtcggcagcgtcagatgtgtataagag |
| OFF9_F | acagGCTCTGACCTTGTTTGTTATTCC |
| | (SEQ ID NO: 38) |
| NGS_VEGFAs2_ | gtctcgtgggctcggagatgtgtataaga |
| OFF9_R | gacagGTGACTCCAAGGCTTTTCG (SEQ |
| | ID NO: 39) |
| NGS_VEGFAs2_ | tcgtcggcagcgtcagatgtgtataagag |
| OFF23_F | acagCTCTTGCCTGTCACGCA (SEQ ID |
| | NO: 40) |
| NGS_VEGFAs2_ | gtctcgtgggctcggagatgtgtataaga |
| OFF23_R | gacagCCTGGAGTT AAGGGT GT CTC |
| | (SEQ ID NO: 41) |
| NGS_VEGFAs2_ | tcgtcggcagcgtcagatgtgtataagag |
| OFF24_F | acagCATCCTTGTATCAGCTGCCT |
| | (SEQ ID NO: 42) |
| NGS_VEGFAs2_ | gtctcgtgggctcggagatgtgtataaga |
| OFF24_R | gacagCCATTCTCAGCCTAAAAGGTA |
| | GA (SEQ ID NO: 43) |

TABLE 5
Dual-Indexing PCR primers for next
generation sequencing

| Name | Sequence (5′ to 3′) |
|---|---|
| NGSIndexFl | AATGATACGGCGACCACCGAGAT |
| | CTACACCTCTCTATTCGTCGGCAG |
| | CGTC (SEQ ID NO: 44) |
| NGS_Index_F2 | AATGATACGGCGACCACCGAGATC |
| | TACACTATCCTCTTCGTCGGCAG |
| | CGTC (SEQ ID NO: 45) |
| NGS_Index_F3 | AATGATACGGCGACCACCGAGATC |
| | TACACGTAAGGAGTCGTCGGCA |
| | GCGTC (SEQ ID NO: 46) |
| NGS_Index_F4 | AATGATACGGCGACCACCGAGATC |
| | TACACACTGCATATCGTCGGCA |
| | GCGTC (SEQ ID NO: 47) |
| NGS_Index_F5 | AATGATACGGCGACCACCGAGATC |
| | TACACAAGGAGTATCGTCGGCA |
| | GCGTC (SEQ ID NO: 48) |
| NGS_Index_F6 | AATGATACGGCGACCACCGAGATC |
| | TACACCTAAGCCTTCGTCGGCAG |
| | CGTC (SEQ ID NO: 49) |
| NGS_Index_F7 | AATGATACGGCGACCACCGAGATC |
| | TACACCGTCTAATTCGTCGGCAG |
| | CGTC (SEQ ID NO: 50) |
| NGS_Index_F8 | AATGATACGGCGACCACCGAGATC |
| | TACACTCTCTCCGTCGTCGGCAG |
| | CGTC (SEQ ID NO: 51) |
| NGSIndexRl | CAAGCAGAAGACGGCATACGAGAT |
| | TCGCCTTAGTCTCGTGGGCTCG |
| | G (SEQ ID NO: 52) |
| NGS_Index_R2 | CAAGCAGAAGACGGCATACGAGAT |
| | CTAGTACGGTCTCGTGGGCTCG |
| | G (SEQ ID NO: 53) |
| NGS_Index_R3 | CAAGCAGAAGACGGCATACGAGAT |
| | TTCTGCCTGTCTCGTGGGCTCGG |
| | (SEQ ID NO: 54) |
| NGS_Index_R4 | CAAGCAGAAGACGGCATACGAGAT |
| | GCTCAGGAGTCTCGTGGGCTCG |
| | G (SEQ ID NO: 55) |
| NGS_Index_R5 | CAAGCAGAAGACGGCATACGAGAT |
| | AGGAGTCCGTCTCGTGGGCTCG |
| | G (SEQ ID NO: 56) |
| NGS_Index_R6 | CAAGCAGAAGACGGCATACGAGAT |
| | CATGCCTAGTCTCGTGGGCTCG |
| | G (SEQ ID NO: 57) |
| NGS_Index_R7 | CAAGCAGAAGACGGCATACGAGAT |
| | GTAGAGAGGTCTCGTGGGCTCG |
| | G(SEQ ID NO: 58) |
| NGS_Index_R8 | CAAGCAGAAGACGGCATACGAGAT |
| | CAGCCTCGGTCTCGTGGGCTCG |
| | G (SEQ ID NO: 59) |

TABLE 6
PCR reagent mixtures

| Amplicon PCR (Sanger, NGS) | | Index PCR (NGS) | |
|---|---|---|---|
| gDNA (5-150 ng/ul) | 1 μl | PCRI amplicon | 1 μl |
| F/R primer mix (5 μM) | 1 μl | F/R primer mix (5 uM) | 1 μl |
| Nuclease free water | 3 μl | Nuclease free water | 3 μl |
| Q5 Hot Start 2x Master | 5 μl | KAPA HiFi HotStart 2x | 5 μl |
| Mix | | Ready Mix | |
| Total | 10 μl | Total | 10 μl |

TABLE 7
PCR thermocycling conditions
Sanger sequencing and generating target DNA for in vitro

| Sten | Tem | Time |
|---|---|---|
| Initial Denaturation | 98° C. | 30 sec |
| 28 cycles | 98° C. | 10 sec |
| | X | 10 sec |
| | 72° C. | 20 sec |
| Final extension | 72° C. | 2 min |
| Hold | 4° C. | |

X = 71C (ACTB), 68C (HEK site 4, PPP1R2Y 65C (VEGFA site 2)

ACTB (NGS)

| Sten | Tem | Time |
|---|---|---|
| Initial Denaturation | 98° C. | 30 |
| 28 cycles | 98° C. | 10 |
| | 71° C. | 10 |
| | 72° C. | 20 |
| Final extension | 72° C. | 5 min |
| Hold | 4° C. | |

HEK site 4 (NGS)-Touchdown PCR

| Sten | Tem | Time | |
|---|---|---|---|
| Initial Denaturation | 98° C. | 30 | |
| 6 cycles | 98° C. | 10 | |
| | 72-67° C. | 10 sec | (−1° C./cycle) |
| | 72° C. | 20 | |
| X cycles | 98° C. | 10 | |
| | 63° C. | 10 | |
| | 72° C. | 20 | |
| Final extension | 72° C. | 5 min | |
| Hold | 4° C. | | |

X = 26 for ON, OFF1, OFF10; X = 29 for OFF3

VEGFA site 2 (NGS)-Touchdown PCR

| Sten | Temn | Time | |
|---|---|---|---|
| Initial Denaturation | 98° C. | 30 sec | |
| 8 cycles | 98° C. | 10 sec | |
| | 72-65° C. | 10 sec | (−1° C./cycle) |
| | 72° C. | 20 sec | |
| X cycles | 98° C. | 10 sec | |
| | 63° C. | 10 sec | |
| | 72° C. | 20 sec | |
| Final extension | 72° C. | 5 min | |
| Hold | 4° C. | | |

X = 20 for ON; X = 27 for OFF9, OFF23, OFF24

Kana HiFi Index PCR conditions

| Sten | Tem | Time |
|---|---|---|
| Initial denaturation | 95° C. | 3 min |
| 10 cycles | 95° C. | 30 |
| | 55° C. | 30 |
| | 72° C. | 30 |
| Final extension | 72° C. | 5 min |
| Hold | 4° C. | |

### Example 2: Mathematical Model

The mathematical model herein is derived as a mathematical model to quantify how increased duration of exposure to active enzymes (SpCas9 or AncBE4max) will lead to an increase in effective “conversion” from final unmodified target DNA to final edited target DNA (indels or base edits) at 72 h after RNP delivery. The goal of this modeling is to allow curve fitting and obtain phenomenological rate constants and is not an attempt to directly model some underlying kinetic processes in cells (i.e. direct conversion from final unmodified to edited target DNA does not biologically occur).

At 72 h after delivery of SpCas9 or AncBE4max, cells initially exposed to active RNP for various durations of time were harvested. Fraction of genomic DNA that contains either an indel or base edit at the target position was determined by Sanger sequencing-based TIDE analysis (Brinkman, E. K., et al. Easy quantitative assessment of genome editing by sequence trace decomposition. Nucleic Acids Research 42, 168 (2014)) or targeted deep sequencing.

The following measurable quantities were defined as:

B(t): Fraction of final edited target DNA after t hours of exposure to active RNPs

A(t): Fraction of final unmodified target DNA after t hours of exposure to active RNPs where A(t)=1−B(t).

An important goal of gene editing is to achieve a high level of desired editing at the endpoint. With temporally confined gene editing, it is important to quantify how increasing the duration of exposure to active enzyme translates to a greater proportion of final edited DNA. In other words, what is the effective rate at which A(t) becomes B(t) as t increases? This can be represented conceptually by the process:

$$A ( t ) {\to }^{k ( t )} B ( t )$$

where k(t) is the rate of change from the final unmodified target DNA to final edited target DNA. k(t) has a dependence on t because the amount of RNP that is able to perform gene editing diminishes over time due to degradation. Therefore, increasing the duration of exposure at later time points should also have diminishing effects on effective A(t) to B(t) conversion. This conversion rate is represented with:

k(t)=kₑe^{-kdt}

where kₑ is the initial “conversion” rate and k_{d} modulates the rate through an exponential decay function to represent the diminishing conversion effects at later time points.

The practical interpretation of kₑ is the rate at which the final unmodified target DNA A(t) changes to final edited target DNA B(t) if the activity duration increases from t=0 to t=Δt. The practical interpretation of k_{d} is the rate at which this change is dampened (assuming an exponential decay process) if it occurs at later time points, which can be attributed predominantly to decreasing concentrations of active RNPs over time due to degradation.

The final model equation for the “conversion” can now be defined from final unmodified to edited target DNA as a function of t:

$$\frac{dB ( t )}{dt} = k ( t ) \times A ( t ) = {k}_{e} {e}^{- kdt} \times ( 1 - B ( t ) )$$

$$B ( 0 ) = 0$$

A closed form solution can be obtained from the previous differential equation with initial conditions:

$$B ( t ) = 1 - e \frac{( {e}^{- {k}_{d} t} - 1 ) \times {k}_{e}}{{k}_{d}}$$

However, because only a proportion of E_{f} of all cells actually receive active RNP, the actual measured proportions for edited vs. unmodified DNA from sequencing is:

B*(t)=E_{f}B(t): measured fraction of final edited target DNA
A*(t)=1−B*(t): measured fraction of final unmodified target DNA

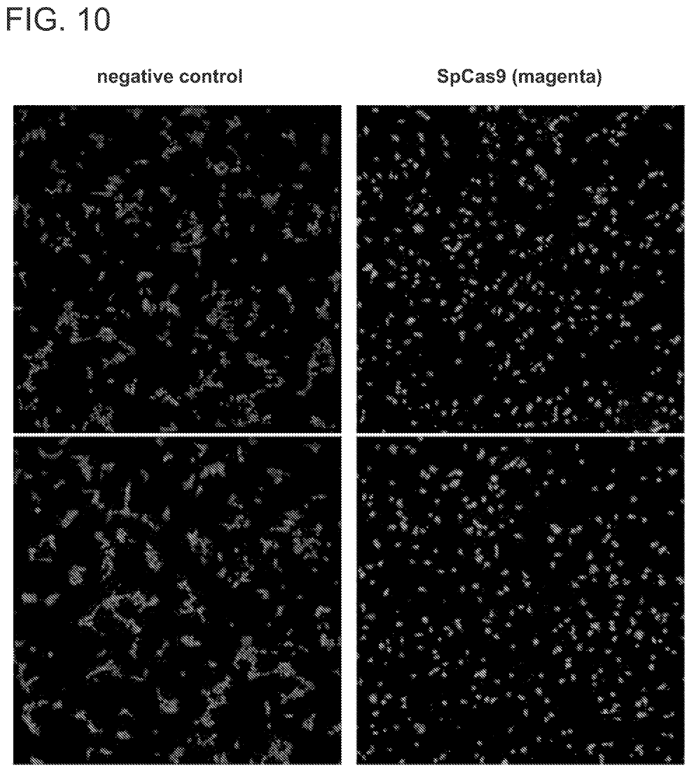

Ef is experimentally determined to be 0.97 from immunofluorescence staining with Cas9 antibody, 1 hour after electroporation (FIG. 10).

Experimental data are fit to B*(t) using non-linear squares optimization. The MATLAB function ‘fit’ was used with the default ‘Trust-Region’ algorithm to perform all fitting.

For SpCas9 indels, kₑ was determined for each locus and k_{d} was determined as a single rate from all loci. The same methodology was used for AncBE4max-mediated base editing and indels.

### Example 3: Photocleavable Guide RNAs Natively Improved Genome Editing Specificity

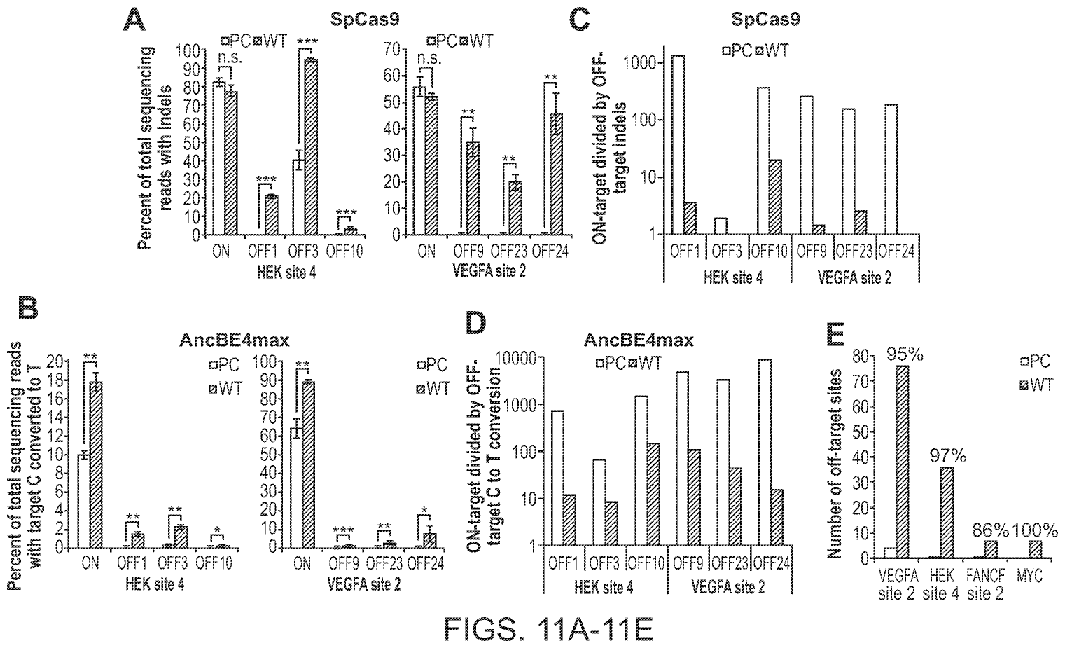

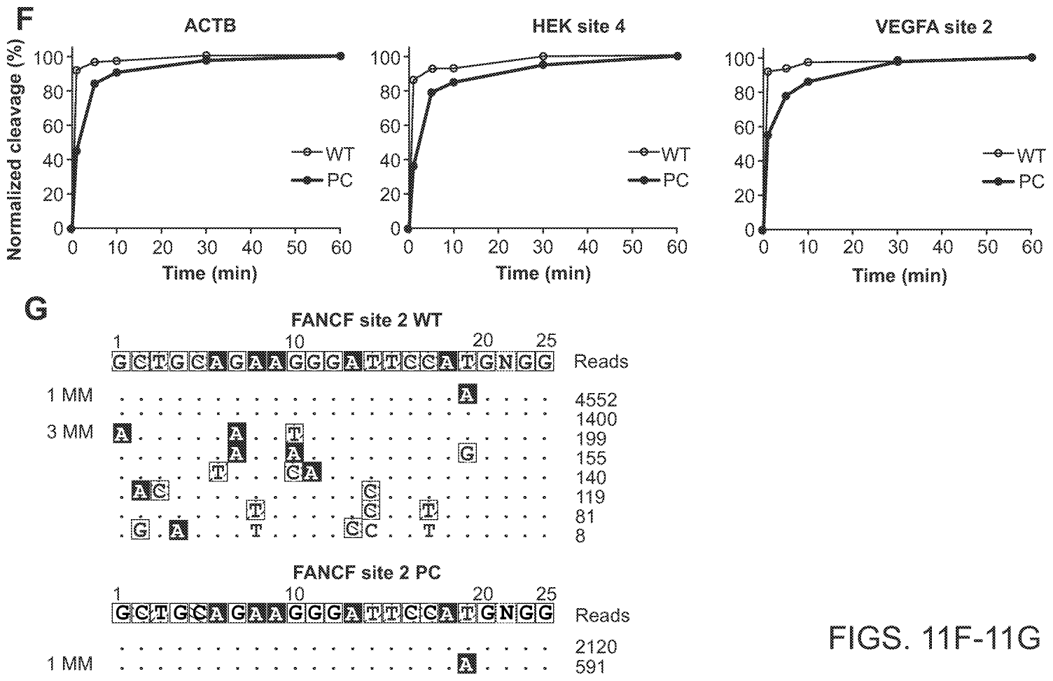

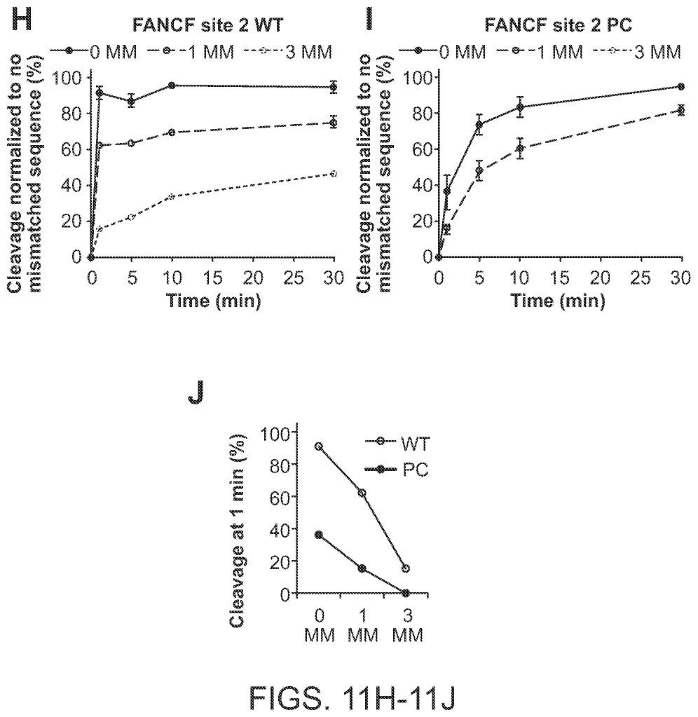

We tested whether inclusion of the photocleavable group to guide RNA would directly enhance Cas9 specificity. We measured indels and cytosine base editing at select off-target sites of VEGFA site 2 and HEK site 4 72 hours after delivery of either Cas9 or AncBE4max with pcRNA to cells. We observed dramatic suppression of Cas9-mediated indels and AncBE4max-mediated base editing at all tested off-target sites (FIGS. 11A and 11B), improving the ratio of on-target to off-target editing by 2 to 9000-fold compared to wild type gRNA (FIGS. 11C and 11D). GUIDE-seq (Tsai et al., Nature Biotechnology 33, 187-197 (2015) at 72 hours after Cas9/pcRNA delivery further revealed that the significantly improved targeting specificity holds genome-wide, with 86% to 100% reduction in the number of detected off-target sites using pcRNA compared to wild type gRNA (FIG. 11E). Proportion of off-target GUIDE-seq reads was also greatly reduced with pcRNA compared to wild type gRNA—comparable to or better than other enhanced specificity Cas9s for the same evaluated target sequences from published datasets.

To understand the mechanism behind the enhanced specificity, we investigated the cleavage kinetics of Cas9/pcRNA using in vitro cleavage assays. For all target sequences tested, the initial cleavage rate was lower using pcRNA compared to wild type gRNA, even though the eventual cleavage efficiency was comparable (FIG. 11F). We further evaluated cleavage of select mismatched target sequences determined from GUIDE-seq results of FANCF site 2 (FIG. 11G). A single mismatch at the PAM-proximal position still led to over 60% cleavage within 1 minute using wild type gRNA, compared to under 20% cleavage using pcRNA (FIG. 11H to 11J). With three mismatches, wild type gRNA still resulted in rapid cleavage whereas pcRNA resulted in almost no activity (FIG. 11J). Together, our results suggest that pcRNA provides specificity enhancement through heightened kinetic control over Cas9 cleavage and increased sensitivity to mismatches. Given that Cas9 is believed to exhibit multiple-turnover activity inside cells (Clarke et al., 2018, Wang et al., 2019) with much shorter dwell times for off-target sequences compared to on-target sequences (Knight et al., 2015, Ma et al., 2016), off-target binding sites would be much less likely to experience cleavage before Cas9/pcRNA dissociation. Decrease in the intrinsic cleavage rate and sensitivity to mismatches are also believed to be mechanisms exhibited by other enhanced-specificity Cas9s (Singh et al., 2018).

### Example 4: Minimum “Dose” of Active Genome Editor Necessary for High Editing Efficiencies

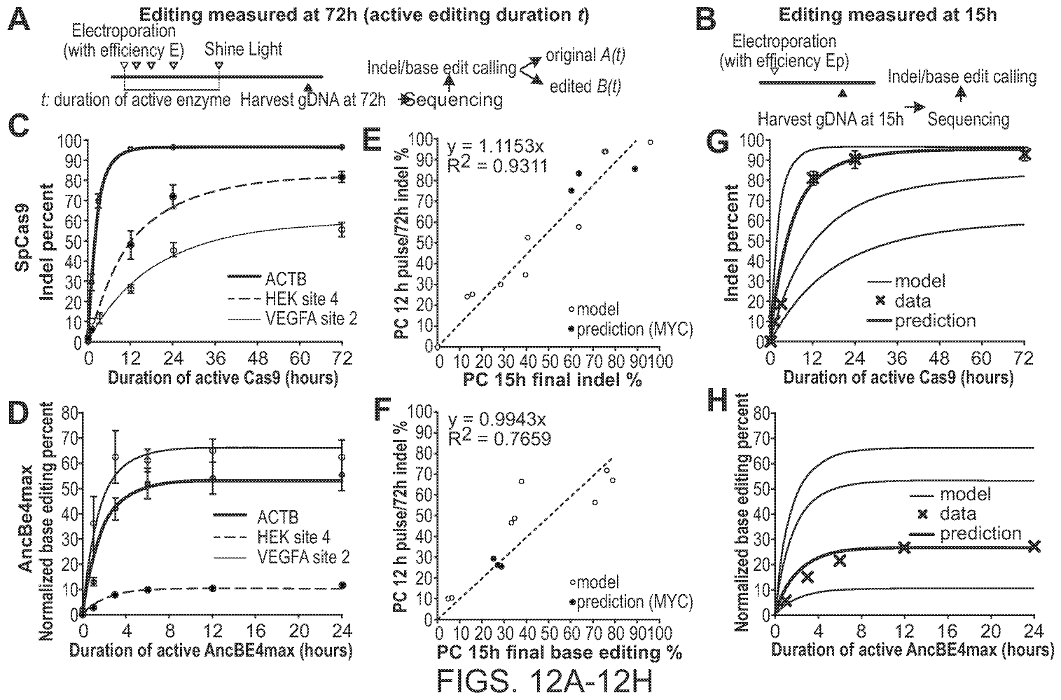

Since genome editing enzymes can be active for days to weeks in cells (Kim et al., 2014, Zuris et al., 2015), characterizing the minimum duration of active enzyme, or ‘dose’ necessary to achieve a desired level of final editing is crucial for balancing high on-target editing with the lowest probability of accruing adverse side-effects (Haapaniemi et al., 2018, Kosicki et al., 2018). We used pcRNA to investigate this question by measuring the effect of varying the deactivation time point on end-point editing percentage evaluated at 72 hours (FIG. 12A). AncBE4max required shorter durations of activity to achieve high editing at end-point, with 2-4 hours sufficient to attain 50-80% of maximum potential, compared to 12-36 hours with Cas9 (FIGS. 12C and 12D).

Because deactivation is complete and sequence independent, we considered that the large variability in dose dependence of editing percentages between target sites can be attributed to heterogeneous, target-dependent genome editing kinetics (Rose et al., 2020). To quantify this difference in editing kinetics, we evaluated editing outcomes of standard Cas9/pcRNA or AncBE4max/pcRNA measured directly at an early 15-hour time point (FIG. 12B). Editing percentage scored at 15 hours was indeed heterogeneous between target sequences, ranging from 10-95% indels for Cas9 and 5-80% base editing for AncBE4max, suggesting that it captures the dynamic range of editing kinetics before genome editing saturation. Crucially, editing percentage at 15 hours was highly correlated with editing percentage, measured at 72 hours, after deactivation at 12 hours. (R² of 0.93 for Cas9 and 0.76 for AncBe4max), confirming that the heterogeneity in dose-dependence of editing percentages between target sites can be attributed to heterogeneous genome editing kinetics (FIGS. 12E and 12F).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A synthetic guide RNA (gRNA) comprising a CRISPR RNA (crRNA), and a trans-activating small RNA (tracrRNA) wherein the crRNA comprises at least one photocleavable linker molecule.

2. The synthetic gRNA of claim 1, wherein the at least one photocleavable linker molecule is located between 10 to 20 nucleotides distal to a protospacer adjacent motif (PAM).

3. The synthetic gRNA of claim 1, wherein the at least one photocleavable linker molecule is capable of reacting with phosphoryl, carboxyl, carbonyl, thiol and amine functionalities.

4. The synthetic gRNA of claim 1, wherein the photocleavable linker molecule comprises one or more 2-nitrobenzyl moieties, alpha-substituted 2-nitrobenzyl moieties, 3,5-dimethoxybenzyl moieties, thiohydroxamic acid, 7-nitroindoline moieties, 9-phenylxanthyl moieties, benzoin moieties, hydroxyphenacyl moieties, or combinations thereof.

5. The synthetic gRNA of claim 4, wherein the photocleavable linker comprises a photocleavable 2-nitrobenzyl linker (PC-Linker).

6. A synthetic CRISPR RNA (crRNA) oligonucleotide comprising at least one photocleavable linker molecule.

7. The synthetic crRNA oligonucleotide of claim 6, wherein the at least one photocleavable linker molecule is capable of reacting with phosphoryl, carboxyl, carbonyl, thiol and amine functionalities.

8. The synthetic crRNA oligonucleotide of claim 6, wherein the photocleavable linker molecule comprises one or more 2-nitrobenzyl moieties, alpha-substituted 2nitrobenzyl moieties, 3,5-dimethoxybenzyl moieties, thiohydroxamic acid, 7-nitroindoline moieties, 9-phenylxanthyl moieties, benzoin moieties, hydroxyphenacyl moieties, or combinations thereof.

9. The synthetic crRNA oligonucleotide of claim 6, wherein the photocleavable linker is a photocleavable 2-nitrobenzyl linker (PC-Linker).

10. A composition comprising an engineered nucleic acid sequence encoding: a clustered regularly interspaced short palindromic repeats (CRISPR)-associated endonuclease, a Cas peptide and at least one guide RNA comprising at least one photocleavable linker molecule.

11. The composition of claim 10, wherein the at least one photocleavable linker molecule is located between 10 to 20 nucleotides distal to a protospacer adjacent motif (PAM).

12. The composition of claim 10, wherein the at least one photocleavable linker molecule is capable of reacting with phosphoryl, carboxyl, carbonyl, thiol and amine functionalities.

13. The composition of claim 10, wherein the photocleavable linker molecule comprises one or more 2-nitrobenzyl moieties, alpha-substituted 2-nitrobenzyl moieties, 3,5-dimethoxybenzyl moieties, thiohydroxamic acid, 7-nitroindoline moieties, 9-phenylxanthyl moieties, benzoin moieties, hydroxyphenacyl moieties, N-hydroxysuccinimidyl-4-azidosalicyclic acid (NHS-ASA), a protective group selected from the group consisting of 9-fluorenylmethoxycarbonyl (Fmoc), 2-(4biphenyl)propyl(2)oxycarbonyl (Bpoc), and derivatives thereof.

14. (canceled)

15. The composition of claim 1, further comprising a sequence encoding a transactivating small RNA (tracrRNA).

16. The composition of claim 1, further comprising at least two or more gRNAs.

17-24. (canceled)

25. A method of deactivating a gene editing agent comprising:
contacting a cell with a composition comprising a gene editing agent, wherein the gene-editing agent comprises a clustered regularly interspaced short palindromic repeats (CRISPR)-associated endonuclease, a Cas peptide and at least one guide RNA (gRNA) comprising at least one photocleavable linker molecule;
subjecting the cell to an electromagnetic radiation, thereby cleaving the at least one gRNA; and,
deactivating the gene-editing agent.

26. (canceled)

27. The method of claim 25, wherein the electromagnetic radiation has a wavelength of about 365 nm.

28. The method of claim 25, wherein the photocleavable linker molecule comprises one or more 2-nitrobenzyl moieties, alpha-substituted 2-nitrobenzyl moieties, 3,5-dimethoxybenzyl moieties, thiohydroxamic acid, 7-nitroindoline moieties, 9-phenylxanthyl moieties, benzoin moieties, hydroxyphenacyl moieties, N-hydroxysuccinimidyl-4-azidosalicyclic acid (NHS-ASA), a protective group selected from the group consisting of 9-fluorenylmethoxycarbonyl (Fmoc), 2-(4biphenyl)propyl(2)oxycarbonyl (Bpoc), and derivatives thereof.

29-32. (canceled)

33. A kit comprising the guide RNA (gRNA) of claim 1.

34. (canceled)
